(19) United States
(12) Patent Application Publication
(10) Pub. No.: US 2008/0155704 A1
(43) Pub. Date: Jun. 26, 2008

(54) Methods and Compositions for Determining in vivo Activity of BiP

(75) Inventors: Gabriel Stavros Panayi, London (GB); Valerie Mary Corrigal, Tadworth Surrey (GB)

Correspondence Address: HOWREY LLP, C/O IP DOCKETING DEPARTMENT, 2941 FAIRVIEW PARK DR, SUITE 200, FALLS CHURCH, VA, 22042-2924, US

(73) Assignee: King's College London, London (GB)

(21) Appl. No.: 11/962,459

(22) Filed: Dec. 21, 2007

Related U.S. Application Data
(60) Provisional application No. 60/876,897, filed on Dec. 22, 2006.

(51) Int. Cl. A01K 67/027; C12Q 1/02; G01N 33/567

(52) U.S. Cl. 800/3; 435/29; 435/7.21

(57) ABSTRACT

The present invention relates to methods for determining the functionality of BiP in vivo, comprising the use of one or more markers. Uses of the markers and kits for determining the functionality of BiP in vivo are also described.

The present application claims the benefit of priority of U.S. Provisional Application No. 60/876,897 which was filed 22 Dec. 2006. The aforementioned application is incorporated by reference in its entirety.

### FIELD OF INVENTION

The present invention relates to inter alia markers that can be used to determine the functionality of the BiP protein in vivo.

### BACKGROUND TO THE INVENTION

BiP is a human molecular chaperone known variously as binding immunoglobulin protein (BiP) or glucose regulated protein (Grp)78 that has anti-inflammatory effect (1). The gene encoding BiP has been cloned and the recombinant human (rhu) protein expressed (WO 00/21995).

Administration of rhuBiP to mice with collagen-induced arthritis (CIA), prevented the induction of experimental arthritis (1). An indication of the anti-inflammatory mechanism of action of BiP, was the finding that T cell clones responsive to rhuBiP produced the cytokines, IL-10, IL-4 and IL-5 (2) and that BiP-stimulated peripheral blood (PB) mononuclear cells (MC) produced high concentrations of IL-10 with concomitant attenuation of TNF-α production. PBMC also produced increased amounts of IL-1R antagonist and soluble TNFRII (3). Cytokines released from PBMC in response to BiP regulate osteoclastogenesis (4, 5).

To reinforce the fact that these extracellular functions of BiP have biological relevance, immunoassay of synovial fluids has revealed that the majority of those from patients with RA contain soluble BiP (3). It is also known that the antigen presenting function of monocytes (MO) is reduced following downregulation of CD86 and HLA-DR expression (3) and that BiP delays and prevents the maturation of purified PBMO into immature dendritic cells (iDC) (6).

In WO02/072133, it is taught that BiP has immunomodulatory properties and the use of BiP in treating or preventing an unwanted immune response is described. Furthermore, the use of BiP in treating auto-immune diseases is disclosed. Human blood mononuclear cells cultured with BiP release IL-10 which may downregulate auto-immune diseases discussed therein—such as rheumatoid arthritis (RA). Moreover, Corrigall et al. (1) teach that BiP can be used to inhibit the development of rheumatoid arthritis and is a candidate for the immunotherapy of this disorder.

WO2006/11720 teaches that BiP can directly modulate bone resorption and maturation of osteoclasts.

In view of the wide variety of clinical applications of BiP, there is a need in the art to be able to monitor the functionality of BiP in vivo.

### SUMMARY OF THE INVENTION

The inventors have discovered that the marker(s) described herein are induced in vitro in peripheral blood in the presence of the protein, BiP. The one or more phenotypic, molecular and functional markers described herein are also expected to indicate that BiP is functional in vivo. The marker(s) may be used to monitor the therapeutic effect of BiP. Accordingly, the presence of one or more markers in a sample (eg. blood) may be indicative that a subject is responsive to BiP.

### SUMMARY ASPECTS OF THE PRESENT INVENTION

In one aspect, there is provided a method for determining the functionality of BiP in vivo, comprising the steps of: (i) providing a sample from a subject; (ii) contacting the sample with BiP; and (iii) determining the presence of a marker (eg. all markers) in the sample, wherein said marker is selected from the group consisting of: (a) an increase in the number of cells expressing indoleamine 2,3-dioxygenase (IDO); (b) an increase in the number of cells expressing CD8CD122; (c) an increase in the number of cells expressing CD4CD25hiCD27hi; (d) an increase in the number of cells expressing CD4CD25hi FoxP3; (e) an increase in the number of cells expressing intracellular CTLA-4; (f) an increase in the number of cells expressing CCR4; (g) a decrease in the number of cells expressing CD86; (h) an increase in the number of cells expressing cell surface CD85j; (i) an decrease in the number of cells expressing CD83; (j) an increase or no change in the number of cells expressing CD14; (k) a decrease in the number of T regulatory cell or dendritic cells expressing HLA-DR or Class II Major Histocompatibility Complex antigen; (l) a decrease in the number of cells expressing CD80; (m) an increase in the number of cells expressing CD40; (n) a lower CD1a mean fluorescent intensity; (O) a higher CD11c mean fluorescent intensity; (p) dendritic cells that do not present antigen and do not stimulate an allogeneic lymphocyte reaction; (q) an increase in the number of T regulatory cell or dendritic cells expressing IL-10; (r) an increase in the number of human T cells cultured in the peritoneum of SCID mice that show IL-4, IL-5 and/or IL-10 expression and/or a decrease in the number of cells expressing interferon-γ; (s) no upregulation of IFNγ producing cells when stimulated with a T cell mitogen; and (t) a decrease in the concentration of circulating IL-6; or a combination comprising at least 2 of said markers; wherein the difference is measured in comparison to a sample or a cell that has not been induced with BiP, and wherein the presence of the marker(s) in the sample is indicative that BiP is functional in vivo. Optionally said group further comprises (u) a decrease or downregulation of IL-8.

In another aspect, there is provided a method for determining the functionality of BiP in vivo, comprising the steps of: (i) providing a sample from a subject; (ii) contacting the sample with BiP; and (ii) determining the presence of a marker (eg. all markers) in the sample, wherein said marker is selected from the group consisting of: (a) an increase in the number of cells expressing indoleamine 2,3-dioxygenase (IDO); (b) an increase in the number of cells expressing CD8CD122; (c) an increase in the number of cells expressing CD4CD25hiCD27hi; (d) an increase in the number of cells expressing CD4CD25hi FoxP3; (e) an increase in the number of cells expressing intracellular CTLA-4; (f) an increase in the number of cells expressing CCR4; (g) a decrease in the number of cells expressing CD80; (h) lower CD1a mean fluorescent intensity; (i) higher CD11c mean fluorescent intensity; (j) dendritic cells that do not present antigen and do not stimulate an allogeneic lymphocyte reaction; (k) an increase in the number of human T cells cultured in the peritoneum of SCID mice that show IL-4, IL-5 and/or IL-10 expression and/or a decrease in the number of cells expressing interferon-γ; (l) no upregulation of IFNγ producing cells when stimulated with a T cell mitogen; and (m) a decrease in the concentration of circulating IL-6; or a combination comprising at least 2 of said markers; wherein the difference is measured in comparison to a sample or a cell that has not been induced with BiP; and wherein the presence of the marker(s) in the sample is indicative that BiP is functional in vivo.

In a further aspect, there is provided a kit for determining the functionality of BiP in vivo comprising a plurality of labelled antibodies, wherein said labelled antibodies specifically hybridise or bind to CD14, CD80, CD83, CD86, CD85j, HLA-DR, CD11, CD1, CD4, CD8, CD25, CD27, CD122, CTLA-4, FoxP3, IL-10, IL-4, IL-5, IL-6, IFNγ, IDO, CCR-4 and CD40.

In a further aspect, there is provided a kit for determining the functionality of BiP in vivo comprising a plurality of labelled antibodies, wherein said labelled antibodies specifically hybridise or bind to CD14, CD80, CD83, CD86, CD85j, HLA-DR, CD11, CD1, CD4, CD8, CD25, CD27, CD122, CTLA-4, FoxP3, IL-10, IL-4, IL-5, IL-6, IFNγ, IDO, CCR-4, CD40 and IL-8.

In a still further aspect, there is provided a kit for determining the functionality of BiP in vivo comprising a plurality of labelled antibodies, wherein said labelled antibodies specifically hybridise or bind to CD80, CD11, CD1, CD4, CD8, CD25, CD27, CD122, CTLA-4, FoxP3, IL-10, IL-4, IL-5, IL-6, IFNγ, IDO and CCR-4.

In another aspect, there is provided the use of a marker for determining the functionality of BiP in vivo, wherein said marker is selected from the group consisting of: (a) an increase in the number of cells expressing indoleamine 2,3-dioxygenase (IDO); (b) an increase in the number of cells expressing CD8CD122; (c) an increase in the number of cells expressing CD4CD25hiCD27hi; (d) an increase in the number of cells expressing CD4CD25hi FoxP3; (e) an increase in the number of cells expressing intracellular CTLA-4; (f) an increase in the number of cells expressing CCR4; (g) a decrease in the number of cells expressing CD86; (h) an increase in the number of cells expressing cell surface CD85j; (i) an decrease in the number of cells expressing CD83; (j) an increase or no change in the number of cells expressing CD14; (k) a decrease in the number of T regulatory cell or dendritic cells expressing HLA-DR or Class II Major Histocompatibility Complex antigen; (l) a decrease in the number of cells expressing CD80; (m) an increase in the number of cells expressing CD40; (n) a lower CD1a mean fluorescent intensity; (O) a higher CD11c mean fluorescent intensity; (p) dendritic cells that do not present antigen and do not stimulate an allogeneic lymphocyte reaction; (q) an increase in the number of T regulatory cell or dendritic cells expressing IL-10; (r) an increase in the number of human T cells cultured in the peritoneum of SCID mice that show IL-4, IL-5 and/or IL-10 expression and/or a decrease in the number of cells expressing interferon-γ; (s) no upregulation of IFNγ producing cells when stimulated with a T cell mitogen; and (t) a decrease in the concentration of circulating IL-6; or a combination comprising at least 2 of said markers; wherein the difference is measured in comparison to a cell that has not been induced by BiP. Optionally said group further comprises (u) a decrease or downregulation of IL-8.

In another aspect, there is provided the use of a marker for determining the functionality of BiP in vivo, wherein said marker is selected from the group consisting of: (a) an increase in the number of cells expressing indoleamine 2,3-dioxygenase (IDO); (b) an increase in the number of cells expressing CD8CD122; (c) an increase in the number of cells expressing CD4CD25hiCD27hi; (d) an increase in the number of cells expressing CD4CD25hi FoxP3; (e) an increase in the number of cells expressing intracellular CTLA-4; (f) an increase in the number of cells expressing CCR4; (g) a decrease in the number of cells expressing CD80; (h) lower CD1a mean fluorescent intensity; (i) higher CD11c mean fluorescent intensity; (j) dendritic cells that do not present antigen and do not stimulate an allogeneic lymphocyte reaction; (k) an increase in the number of human T cells cultured in the peritoneum of SCID mice that show IL-4, IL-5 and/or IL-10 expression and/or a decrease in the number of cells expressing interferon-γ; (l) no upregulation of IFNγ producing cells when stimulated with a T cell mitogen; and (m) a decrease in the concentration of circulating IL-6; or a combination comprising at least 2 of said markers; and wherein the presence of the marker(s) in the sample is indicative that BiP is functional in vivo.

There is also provided a method for generating a regulatory T-cell in vivo or in vitro comprising the use of BiP.

A further aspect provides a method for generating a tolerogenic dendritic cell in vivo or in vitro comprising use of BiP.

In a further aspect, there is provided the use of BiP for generating regulatory T-cells in vivo or in vitro.

In another aspect, there is provided the use of BiP for generating tolerogenic dendritic cells in vitro or in vivo.

### PREFERRED EMBODIMENTS

In one embodiment, the subject has been administered with BiP.

In one embodiment, the cell that has not been induced with BiP is obtained or obtainable from the subject before the subject has been administered with BiP.

In one embodiment, the presence of one or more markers in the sample may be determined by transferring the sample intraperitoneally into severe combined immunodeficient (SCID) mice.

In one embodiment, the cell expressing the marker set forth in (b), (c), (d) (e), (f), (g), (h), (i), (j), (k), (l) and/or (m) is a regulatory T-cell or a dendritic cell.

In one embodiment, the cell expressing the marker set forth in (b), (c), (d) (e), (f), (g) and/or (h) is a regulatory T-cell or a dendritic cell.

In one embodiment, the regulatory T-cell is obtained or obtainable by differentiation of a dendritic cell into a T-cell in the presence of BiP.

In one embodiment, the cells inhibit autologous T-cells from responding to anti-CD3 stimulation.

In one embodiment, marker (f) is expressed on a T suppressor cell.

In one embodiment, marker (g), (i), (k) and/or (l) is expressed on an early immature dendritic cell.

In one embodiment, marker (g) is expressed on an early immature dendritic cell.

In one embodiment, marker (h) or (m) is expressed on an immature dendritic cell.

In one embodiment, marker (g), (i) and/or (j) is expressed on a mature dendritic cell.

In one embodiment, the mature dendritic cell is a mature tolerogenic dendritic cell.

In one embodiment, a difference in fluorescence intensity of marker (n) and/or marker (O) is observed on a dendritic cell.

In one embodiment, the dendritic cell is a monocycte derived dendritic cell.

In one embodiment, the monocycte derived dendritic cell is obtained or obtainable by culturing peripheral blood monocytes in the presence of one or more cytokines, optionally in the presence of lipopolysaccharide.

In one embodiment, the cytokine is GM-CSF and/or IL-4.

In one embodiment, marker (r) is measured about 11 days after contacting the sample with BiP.

In one embodiment, marker (l) is measured about 11 days after contacting the sample with BiP.

In one embodiment, the antibody used in the kit is a monoclonal antibody,

In one embodiment, the antibody label is a florescent label.

In one embodiment, the kit comprises concanavalin A.

In one embodiment, the kit comprises anti-CD3 and/or CD28 beads. This had the advantage of being more convenient for functional tests. Thus in one embodiment, the kit comprises anti-CD3 and/or CD28 beads for functional tests.

In one embodiment, the kit comprises immunofluorescent buffer and/or saponin solution.

In one embodiment, the method for generating a regulatory T-cell in vivo or in vitro comprises culturing a T-cell in the presence of BiP.

In another embodiment, the method for generating a regulatory T-cell in vivo or in vitro comprises adding an autologous or an allogeneic T cell to a BiP treated dendritic cell culture.

In another embodiment, the method for generating a tolerogenic dendritic cell in vivo or in vitro comprises adding BiP to monocytes in the presence of one or more cytokines, followed by maturation.

### DESCRIPTION OF THE FIGURES

FIG. 1

IL-10 production by dendritic cells. Peripheral blood monocyte derived dendritic cells were cultured in GM-CSF and IL-4 for 7 days for mDC, lipopolysaccharide was added for the final 48 hours of culture. The Il-10 was assayed by ELISA.

FIG. 2

Intracellular expression of indoleamine 3,2-dioxygenase (IDO). DC matured in the presence and absence of BiP, were investigated for the production of IDO. Two representative experiments are shown (A, B, E and C, D, F). A-D show dot plot analysis of the DC. E and F are histograms of the same figures with A and C (solid) and B and D (open)

FIG. 3

(a) Inhibition of autologous T cell responses to anti-CD3 antibody. Two representative experiments show that allogeneic T cells cultured with dendritic cells differentiated in the presence and absence of BiP when placed in culture with autologous irradiated peripheral blood mononuclear cells and fresh T cells (1:1:1 ratio) will inhibit the responses of the fresh T cells to anti-CD3 antibody. The % inhibition of responses are shown.

(b) Inhibition of autologous T cells responses to anti-CD3. Post 4 day co-culture with allogeneic (allo) or autologous (auto) DC, derived in the presence of BiP, T cells inhibit the response of fresh autologous T cells to anti-CD3 antibody. The mean and standard deviation of five (auto) and three (allo) experiments is shown.

(c) Expression of regulatory T cell phenotype. A FACScan dot plot showing CD4+CD25hiCD27hi expressing cells after 4 days contact between T cells with immature dendritic cells (iDC) or mature dendritic cell (mDC) either untreated or post BiP treatment.

FIG. 4

CCR4 and CCR5 expression by T cells. The expression of CCR4 was significantly raised in three experiments using peripheral blood mononuclear cells where cells were incubated, with or without BiP, for four days only. CCR5 expression was slightly downregulated but failed to reach significance

FIG. 5

Cells from mice given BiP 11 days previously and then cultured overnight with con A (mitogen). FIG. 5 shows there is a significant reduction in IFN production by the BiP treated mouse cells.

FIG. 6 shows details of genes affected by BiP.

FIG. 7 shows IL-8 production by dendritic cells.

FIG. 8 shows that monocyte interaction with cell surface expressed BiP can reduce production of IL-8.

FIG. 9 shows downregulation of IL-8.

FIG. 10: BiP alters expression of indolamine-2,3 dioxygenase, costimulatory and regulatory molecules by dendritic cells: Peripheral blood monocytes were cultured with GM-CSF and IL-4 for 7d in the presence or absence of BiP (20 μg/ml). Lipopolysaccharide (LPS)(500 ng/ml) was added for the final 48 h to give mature dendritic cells (mDC). (A) Flow cytometry was used to detect intracellular expression of indoleamine 2,3-dioxygenase (IDO) after DC maturation in 9 different experiments. (B) BiP induced a dose dependent change in the expression of CD14 and CD86 on mature DC ((BA) isotype control, (BB) 0, (BC) 0.5, (BD) 1.0, (BE) 5.0 and (BF) 20 μg/ml BiP). Expression of CD83 (C), HLA-DR (D) or CD85j (E) (open histogram) or isotype control (solid histogram) by mDC or mDC(BiP) as indicated. Representative examples of 5/6 experiments

FIG. 11: Co-culture of T cells with BiP-treated dendritic cells (DC) upregulates regulatory markers. T cells were co-cultured with autologous mature DC (mDC) or BiP-treated mDC (mDC(BiP) for 4d. Immunofluorescence and flow cytometry showed % positive expression of (A) CD27hi CD25hi cells, when gated on CD4+ cells, and (B) CTLA-4+CD4+ cells. (C) 1 methyl tryptophan (1MT) (200 μM) was used to inhibit IDO function in the DC/T cell co-cultures. After 4d intracellular CTLA-4 in CD4+ T cells was investigated by immunofluorescence and flow cytometry. (D) Addition of IDO inhibitor, 1 methyl tryptophan (200 μM)(1MT) to co-cultures of T cells with autologous mDC or mDC(BiP), increased proliferation by the T cells in an allogeneic response as measured by uptake of tritiated thymidine and recorded as cpm. Mean ±standard deviation of three experiments is shown.

FIG. 12: Co-culture with BiP-treated dendritic cells (DC) leads T cells to have regulatory function. After 4d pre-incubation with either autologous or allogeneic DC, or DC(BiP), T cells were washed and set up in autologous cultures with irradiated peripheral blood mononuclear cells (PBMC) and responder T cells (1:1:1 ratio) (105 cells each/well) in the presence or absence of anti-CD3 antibody (1/2000 dilution). (A) % inhibition of the responder T cell proliferation to anti-CD3 antibody caused by T cells preincubated in either an autologous (Auto) or allogeneic (Allo) system (autologous, n=5, allogeneic n=3). (B) a representative experiment showing an autologous experiment using Δ counts per minute (Δcpm) (stimulated-unstimulated cpm) following stimulation with anti-CD3 antibody to irradiated PBMC (irMC) or responder T cells (rT) alone and cultures with irMC+rT+T cells prestimulated (pT) by autologous DC populations as indicated; (C) mean ±standard deviation of unstimulated and stimulated cultures from a representative experiment where autologous irMC+rT+pT, previously cocultured with allogeneic DC populations as indicated, were present in ratio 1:1:1.

FIG. 13: The effect of IL-10 and MAPK inhibitors on DC maturation:

Allogeneic responses by T cells in culture with irradiated (3,000 rads) early DC (eDC), after only 3d in culture with GM-CSF and IL-4 in the presence or absence of BiP (20 μg/ml) at a ratio of 10:1 PBMC:DC (105:104 cells/well). The results are shown as an allogeneic proliferative response (A) following addition of neutralizing anti-IL-10 ( 1/100 dilution) antibody and (B) the pre-treatment (2 h) of monocytes (MO) with p38 inhibitor, SB203580 (SB)(10 μM) or the ERK1/2 inhibitor, PD 98059 (PD)(10 μM), prior to the addition of GM-CSF and IL-4 to the cultures. The proliferation of PBMC to early DC following (C) the addition of BiP 24 h post addition of GM-CSF and IL-4. Proliferation was measured by uptake of tritiated thymidine and recorded as counts per minute (cpm). ELISA was used to detect IL-10 (D) in the culture supernatants and (E) by 7d cultured iDC/iDC(BiP) and fully matured mDC/mDC(BiP) differentiated as described in following 7d culture in GM-CSF and IL-4 with LPS (500 ng/ml) added for the final 2d.

FIG. 14: BiP alters intracellular expression of IL-4 and surface HLA-DR on T cells following intraperitoneal injection into SCID mice.

Human peripheral blood mononuclear cells (50.106/mouse), either with concomitant BiP (100 μg/mouse) or with PBS/vehicle, were injected into the peritoneal cavity of SCID mice. After 11d the cells were flushed from the peritoneal cavity and investigated by flow cytometry. (A) Intracellular expression of IL-4 was measured 3 h post ex vivo stimulation with PMA (10 ng/ml) and ionomycin (1 μg/ml) in the presence of monensin. (B) surface expression of HLA-DR CD4+ cells. The mean and standard deviation of 3 experiments are shown

FIG. 15: BiP alters costimulatory molecule expression and cytokine profile of human rheumatoid synovium transplanted into SCID mice.

Small pieces of human synovial tissue from patients with rheumatoid arthritis were transplanted into SCID mice. After successful engraftment, BiP (10 μg/animal) or human serum albumin (HSA) (10 μg/animal) were administered intravenously and the tissue removed for histological testing after 12d. In each of the photomicrographs the left-hand figures show explants of synovial membrane taken from mice injected with the control protein, HSA, while the right-hand figures shows explants taken from mice injected with BiP. CD86, HLA-DR, TNFα and IL-6 expression was measured by immunohistology. (B) the weight of explants retrieved from the mice treated as indicated after 12d. In this experiment anti-IL-10 antibody or an isotype control antibody were administered to the mice with the BiP or HSA.

### DETAILED DESCRIPTION OF THE INVENTION

### BiP

As used herein, the term “BiP” refers to the 78 kD endoplasmic reticulum chaperone protein as disclosed in WO 00/21995. In one embodiment, the BiP polypeptide has the amino acid sequence as shown in appendix 2 of WO00/21995 at page 23. In one embodiment, the BiP protein has the amino acid sequence given in WO 00/21995 as SEQ ID NO. 1 or SEQ ID NO. 2. In one embodiment, the BiP sequences used herein are devoid of tags such as the 6H is tags present in the polypeptides referred to above. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

In one embodiment, the BiP protein is encoded by the nucleotide sequence given in WO 00/21995 as SEQ ID NO. 3. The nucleotide sequence may be DNA or RNA of genomic, synthetic or recombinant origin e.g. cDNA. The nucleotide sequence may be double-stranded or single-stranded whether representing the sense or antisense strand or combinations thereof. The nucleotide sequence may be prepared by use of recombinant DNA techniques (e.g. recombinant DNA). In particular, methods for the expression of BiP in bacterial hosts—such as E. Coli—and purification of the recombinant protein are disclosed in WO 00/21995. As will be appreciated by the person skilled in the art, BiP may also be over-expressed in other heterologous hosts—such as mammalian or insect cells. The nucleotide sequence may be the same as the naturally occurring form, or may be a fragment, homologue, variant or derivative thereof.

### Sample

As used herein, the term “sample” has its natural meaning.

A sample may be any physical entity from a subject—such as mammal (eg. a human or an animal)—in which one or more of the markers described herein are detected or identified.

The sample from a subject may be or may be derived from biological material—such as tissues, cells or fluids.

The sample may be from a subject that has been administered with BiP or a subject that is to be administered with BiP.

The sample may be or may be derived from bone marrow or a component thereof.

The sample may be or may be derived from blood or a component thereof.

In one embodiment, the sample may be or may be derived from peripheral blood monocytes contained in blood.

In another embodiment, the sample may be or may be derived from peripheral blood monocytes extracted from blood.

In another embodiment, the sample may be or may be derived from peripheral blood monocytes purified from blood.

In another embodiment, the sample may be or may be derived from dendritic cells from peripheral blood monocytes.

In another embodiment, the sample may be or may be derived from T cells (eg. regulatory T-cells) generated from peripheral blood monocytes.

Peripheral blood monocytes may be cultured in the presence of one or more cytokines—such as GM-CSF and/or IL-4—optionally in the presence of lipopolysaccharide in order to obtain dendritic cells which express one or more of the markers described herein.

### Functionality of BiP

As used herein, the term “functionality of BiP” refers to the expression and/or activity of BiP.

It is desirable to determine the functionality of BiP in vivo in order to establish if a subject that has been administered with BiP shows a response thereto. The presence of one or more markers in a sample from the subject is indicative that the subject may be responsive to BiP. The absence of one or more (eg. all) markers in a sample from the subject is indicative that the subject may not be responsive or may have a decreased responsiveness to BiP.

It is also desirable to monitor a subject's response to BiP therapy. The presence of one or markers in a sample from the subject may be indicative that the subject is responsive to BiP during BiP therapy. The absence of one or more (eg. all) markers in a sample from the subject is indicative that the subject may not be responsive or may have a decreased responsiveness to BiP therapy.

Determining the functionality of BiP in vivo may be important for a number of applications.

By way of example, this may provide an insight into whether or not the subject is responsive to BiP. If the subject is responsive, then BiP (or a pharmaceutical composition comprising the same) may be administered in order to treat or prevent one or more diseases. If the subject is not responsive, then it is unlikely that BiP will have any effect in treating or preventing one or more diseases and so BiP may not be administered.

Accordingly, it may be possible to predict subjects that are or are not responsive to BiP (or a pharmaceutical composition comprising the same). This may be important when BiP responsive subjects are required (eg. for clinical trials) or when BiP non-responsive subjects are required (eg. to determine why the subject is not responsive to BiP).

By way of further example, determining the functionality of BiP in vivo may be important in clinical trials in order to study the efficacy of BiP (or a pharmaceutical composition comprising the same). It may not only be possible to determine if the subject is responsive or non-responsive to BiP at the start of the trials, but it may also be possible to determine the responsiveness to BiP during the clinical trial and even in response to different doses and/or administration regimes of BiP.

Determining the functionality of BiP in vivo may also be important during renal transplantation. In this regard, BiP (or a pharmaceutical composition comprising the same) may be administered to a subject in order to prevent or reduce the risk of rejection of the renal transplant. In this regard, the subject may be monitored in order to establish that the subject is responsive or non-responsive to BiP.

Determining the functionality of BiP in vivo may also be important during the use of BiP for the treatment of diseases—such as psoriasis, inflammatory bowel disease (Crohn's disease or ulcerative colitis) or diabetes mellitus (eg. juvenile onset type 1 diabetes mellitus). In this regard, BiP (or a pharmaceutical composition comprising the same) may be administered to a subject in order to prevent or treat the disease. In this regard, the subject may be monitored in order to establish that the subject is responsive, more responsive, non-responsive or less responsive to BiP.

In the event that a non-responsive or less-responsive subject is identified, it may be possible to alter the formulation of BiP and/or to alter the method of administration of BiP and/or to alter the dosage of BiP in order to render the subject responsive to BiP.

In one embodiment, the functionality of BiP in vivo for normal or standard readings for the one or more markers described herein may be determined. This may be accomplished by testing for the presence/absence of the one or more markers in a sample that has not been contacted with BiP.

In another embodiment, normal or standard readings for the one or more markers in a sample may be measured in a subject before the subject is administered with BiP. Accordingly, the presence/absence of one or more markers following the administration of BiP to the subject may be compared with the presence/absence of one or markers in the subject before the administration of BiP. The presence or absence of the one or more markers in the sample may therefore be determined in a subject with a disease that is to be treated with BiP.

In another embodiment, the presence of one or more markers in the sample may be determined by transferring the sample (eg. intraperitoneally) into severe combined immunodeficient (SCID) mice and assessing the presence of one or more markers therein.

Differences between the presence and absence of the one or more markers may be used to establish the functionality of BiP in vivo, The presence of one or markers described herein may be indicative that BiP is functional in the subject. The absence of one or markers described herein may be indicative that BiP is non-functional in the subject.

The markers may even be tailored to evaluate the response of a subject to a particular therapeutic treatment regime and may be used in animal studies, in clinical trials, or in monitoring the treatment of an individual patient. The methods may be repeated on a regular basis.

Diseases that may be treated with BiP (or a pharmaceutical composition comprising the same) include, but are not limited to, rheumatoid arthritis (as described in WO00/21995), unwanted immune responses—such as auto-immune diseases (eg. type 1 diabetes, thyroiditis, multiple sclerosis, systemic lupus erythematosus, Crohn's disease viral or autoimmune hepatitis) (as described in WO02/072133) and bone loss (as described in WO2006/11720).

### Marker

The inventors have discovered that the marker(s) described herein are induced in vitro in human peripheral blood in the presence of BiP. The existence of this panel of phenotypic, molecular and/or functional markers is expected to indicate that BiP is functional in vivo. Accordingly, the presence of the one or more markers in a sample (eg. blood) may be indicative that the subject is responsive to BiP.

In one embodiment, the marker is an increase in the number of cells expressing indoleamine 2,3-dioxygenase (IDO). IDO is an enzyme involved in tryptophan catabolism. In another embodiment, the marker is an increase in the number of cells expressing intracellular IDO. In another embodiment, the marker is an increase in the number of dendritic cells (eg. matured dendritic cell) expressing IDO.

IDO may be measured using intracellular staining for flow cytometry. The change in the number of cells expressing indoleamine 2,3-dioxygenase (IDO) may be measured by comparing the number of cells with cells that have not been induced by BiP.

As described herein, IDO is important in tolerogenic dendritic cells. Until now very few molecules have been shown to upregulate IDO without extensive manipulation in vitro.

In one embodiment, the marker is an increase in the number of cells expressing the protein markers CD8CD122. In another embodiment, the marker is an increase in the number of cells expressing the protein markers CD8CD122 on a T-cell (eg. a T regulatory cell and/or a dendritic cell).

In one embodiment, the marker is an increase in the number of cells expressing the protein markers CD4CD25hiCD27hi. In another embodiment, the marker is an increase in the number of cells expressing the protein markers CD4CD25hiCD27hi on a T-cell (eg. a T regulatory cell and/or a dendritic cell).

In one embodiment, the marker is an increase in the number of cells expressing the protein markers CD4CD25hi FoxP3. In another embodiment, the marker is an increase in the number of cells expressing the protein markers CD4CD25hi FoxP3 on a T-cell (eg. a T regulatory cell and/or a dendritic cell).

In one embodiment, the marker is an increase in the number of cells expressing the protein marker CTLA-4. In another embodiment, the marker is an increase in the number of cells expressing the protein marker CTLA-4 on a T-cell (eg. a T regulatory cell and/or a dendritic cell).

The change in the number of cells expressing the protein markers CD8CD122, CD4CD25hiCD27hi, CD4CD25hi FoxP3 and/or CTLA-4 may be measured by comparing the number of cells expressing the protein with cells that have not been induced by BiP.

In one embodiment, the marker is an increase in the number of cells expressing the protein marker CCR4. In another embodiment, the marker is an increase in the number of cells expressing the protein marker CCR4 on a T cell (eg. a T-suppressor cell).

The change in expression of the protein marker CCR4 may be measured by comparing the number of cells expressing the protein with cells that have not been induced by BiP.

In one embodiment, the marker is a decrease in the number of cells expressing the protein marker CD86. In another embodiment, the marker is a decrease in the number of cells expressing the protein marker CD86 on a BIP induced dendritic cell. In another embodiment, the marker is a decrease in the number of cells expressing the protein marker CD86 on a BIP induced early immature dendritic cell. In another embodiment, the marker is a decrease in the number of cells expressing the protein marker CD86 on a BIP induced mature dendritic cell.

The change in expression of the protein marker CD86 may be measured by comparing the number of cells expressing the protein with cells that have not been induced by BiP.

In one embodiment, the marker is an increase in the number of cells expressing the protein marker CD85j (eg. cell surface CD85j). In another embodiment, the marker is an increase in the number of cells expressing the protein marker CD85j on a BiP induced dendritic cell. In another embodiment, the marker is an increase in the number of cells expressing the protein marker CD85j on a BIP induced immature dendritic cell.

In one embodiment, the marker is a decrease in the number of cells expressing the protein marker CD83. In another embodiment, the marker is a decrease in the number of cells expressing the protein marker CD83 on a dendritic cell. In another embodiment, the marker is a decrease in the number of cells expressing the protein marker CD83 on a BiP induced early immature dendritic cell. In another embodiment, the marker is a decrease in the number of cells expressing the protein marker CD83 on a BiP induced mature dendritic cell.

In one embodiment, the level of surface expression of the protein marker CD14 is maintained. In another embodiment, the level of surface expression of the protein marker CD14 on a BIP induced dendritic cell is maintained. In another embodiment, the level of surface expression of the protein marker CD14 on a BiP induced mature dendritic cell is maintained.

In one embodiment, the marker is a decrease in the number of cells expressing the protein marker HLA-DR or Class II Major Histocompatibility Complex antigen(s). In another embodiment, the marker is a decrease in the number of cells expressing the protein marker HLA-DR on a BIP induced dendritic cell. In another embodiment, the marker is a decrease in the number of cells expressing the protein marker HLA-DR on a BiP induced early immature dendritic cell.

Suitably, this marker causes a decreasing intensity of expression.

In one embodiment, the marker is a decrease in the number of cells expressing the protein marker CD80. In another embodiment, the marker is a decrease in the number of cells expressing the protein marker CD80 on a BIP induced dendritic cell. In another embodiment, the marker is a decrease in the number of cells expressing the protein marker CD80 on a BiP induced early immature dendritic cell.

In one embodiment, the marker is an increase in the number of cells expressing the protein marker CD40. In another embodiment, the marker is an increase in the number of cells expressing the protein marker CD40 on a BIP induced dendritic cell. In another embodiment, the marker is an increase in the number of cells expressing the protein marker CD40 on a BiP induced immature dendritic cell.

The change in the number of cells expressing of CD85j, CD83, CD14, HLA-DR, CD80 and/or CD40 may be measured by comparing the number of cells expressing the protein with cells that have not been induced by BiP.

In some cases, the change in the number of cells expressing CD40, CD86, CD80, CD85j and/or CD14 may be time dependent. Accordingly, the marker may be detectable after 1, 2, 3, 4, 5, 6, or 7 or more days.

In one embodiment, the dendritic cells expressing one or more of the markers described herein may be used to generate regulatory T-cells. The regulatory T-cells may be obtained or obtainable by differentiating T-cells in the presence of dendritic cells that have been exposed to BiP. These T-cells are capable of inhibiting autologous T cells from responding to anti-CD3 stimulation (similar to mitogenic stimulation). This is unexpected since BiP was not present in the final cultures. Without being bound by any particular theory, it appears that BiP modifies the dendritic cell to alter T cell development after direct cell contact.

In one embodiment, the marker is a lower CD11a mean fluorescent intensity. In another embodiment, the marker is a lower CD11a mean fluorescent intensity in a dendritic cell (eg. a monocyte derived dendritic cell).

In one embodiment, the marker is a higher CD 11c mean fluorescent intensity. In another embodiment, the marker is a higher CD 11c mean fluorescent intensity in a dendritic cell (eg. a monocyte derived dendritic cell).

The change in mean fluorescent intensity may be measured by comparing the mean fluorescent intensity with cells (eg. dendritic cells—such as monocyte derived dendritic cells) that have not been induced by BiP.

In one embodiment, the marker is a dendritic cell that does not present antigen and is therefore unable to stimulate an allogeneic lymphocyte reaction as compared to a dendritic cell that has not been induced by BiP.

In one embodiment, the marker is an increase in the number of cells expressing IL-10. In another embodiment, the marker is an increase in the number of cells expressing IL-10 expression on a dendritic cell (eg. a monocyte derived dendritic cell).

The change in IL-10 expression may be measured by comparing the number of cells expressing IL-10 with cells (eg. dendritic cells—such as monocyte derived dendritic cells) that have not been induced by BiP.

In another embodiment, the marker is an increase in the amount of IL-10 secreted by cells. In another embodiment, the marker is an increase in the amount of IL-10 secreted by a dendritic cell (eg. a monocyte derived dendritic cell).

In one embodiment, the marker is an increase in the number of human T cells cultured in the peritoneum of SCID mice that show IL-4, IL-5 and/or IL-10 expression and/or a decrease in the number of cells expressing interferon-γ.

In one embodiment, the marker is the absence of upregulation of IFNγ producing cells when stimulated with a T cell mitogen—such as concanavalin A. This is unexpected since these cells would be expected to upregulate IFNγ.

In one embodiment, the marker is a down regulation of circulating IL-6.

In one embodiment, the marker is not an increase in the number of cells expressing the protein CD85j (eg. cell surface CD85j). In another embodiment, the marker is not an increase in the number of cells expressing the protein CD85j on a BiP induced dendritic cell. In another embodiment, the marker is not an increase in the number of cells expressing the protein CD85j on a BIP induced immature dendritic cell.

In one embodiment, the marker is not a decrease in the number of cells expressing the protein CD83. In another embodiment, the marker is not a decrease in the number of cells expressing the protein CD83 on a BIP induced dendritic cell. In another embodiment, the marker is not a decrease in the number of cells expressing the protein CD83 on a BiP induced early immature dendritic cell. In another embodiment, the marker is not a decrease in the number of cells expressing the protein CD83 on a BiP induced mature dendritic cell.

In one embodiment, the marker is not an increase in the number of cells expressing the protein CD14. In another embodiment, the marker is not an increase in the number of cells expressing the protein CD14 on a BIP induced dendritic cell. In another embodiment, the marker is not an increase in the number of cells expressing the protein CD14 on a BiP induced mature dendritic cell.

In one embodiment, the marker is a not decrease in the number of cells expressing the protein HLA-DR. In another embodiment, the marker is not a decrease in the number of cells expressing the protein HLA-DR on a BIP induced dendritic cell. In another embodiment, the marker is not a decrease in the number of cells expressing the protein HLA-DR on a BiP induced early immature dendritic cell.

In one embodiment, the marker is not an increase in the number of cells expressing the protein CD40. In another embodiment, the marker is not an increase in the number of cells expressing the protein CD40 on a BIP induced dendritic cell. In another embodiment, the marker is not an increase in the number of cells expressing the protein CD40 on a BiP induced immature dendritic cell.

In one embodiment, the marker is not a decrease in the number of cells expressing the protein CD86. In another embodiment, the marker is a not decrease in the number of cells expressing the protein CD86 on a BIP induced dendritic cell. In another embodiment, the marker is not a decrease in the number of cells expressing the protein CD86 on a BIP induced early immature dendritic cell. In another embodiment, the marker is not a decrease in the number of cells expressing the protein CD86 on a BIP induced mature dendritic cell.

The results of BiP administration for the markers described herein can be expressed as: absolute number of cells in a sample, percentage of cells bearing that marker in a sample or the degree of expression (ie. mean fluorescence intensity).

Suitably, the one or more markers may be detected by immunofluorescent staining using labelled conjugated monoclonal antibodies that recognize the markers described herein. FACS analysis may then be used to analyse the cells.

In some embodiments the marker may be or may comprise IL-8. BiP downregulates IL-8 production.

The marker or markers may suitably be selected from biomarkers IDO, CD86, 11c, 1a, 83, 14, HLA-DR CD85j. Markers in this subset all relate to the differentiation of dendritic cells in vitro. These are cells that are cultured in cytokines to drive cell differentiation and the high and low expression stated are in comparison with those cells grown in the same conditions without BiP.

The HLA-R and CD86 are preferred biomarkers of the invention. One reason is that the model which is the nearest we can get to human in vivo, where the RASM is transplanted into SCID mice (with no T/B cells), there is a downregulation of HLA-DR and CD86, (TNF and IL-6), by BiP.

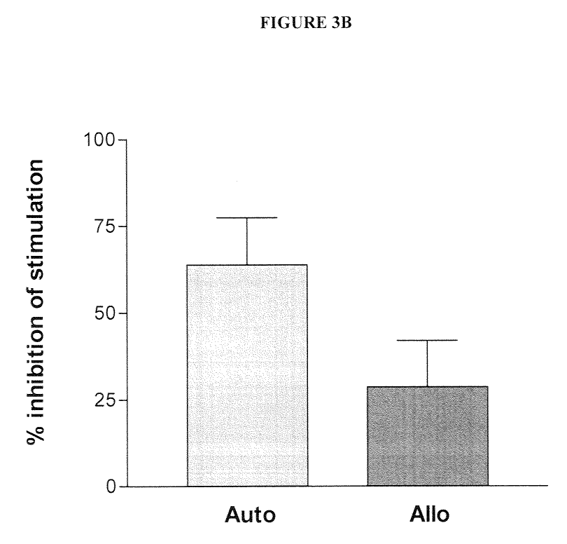

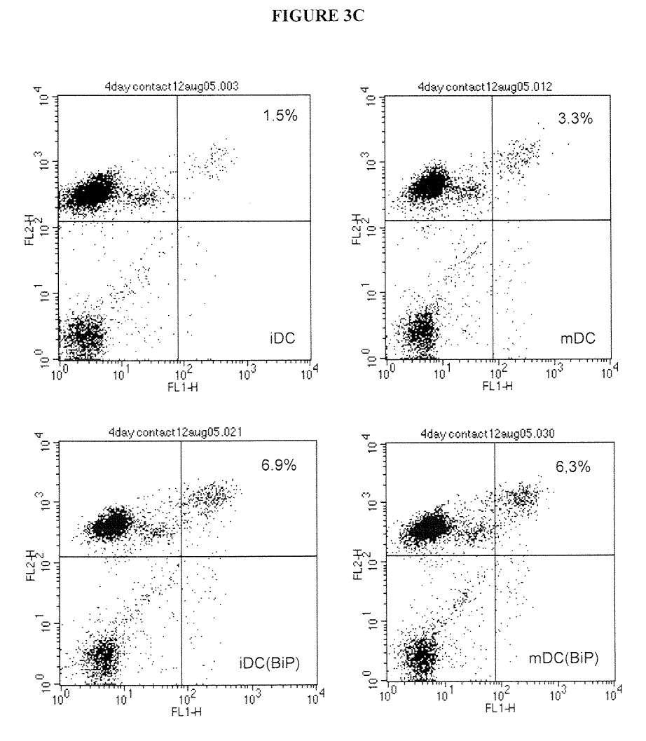

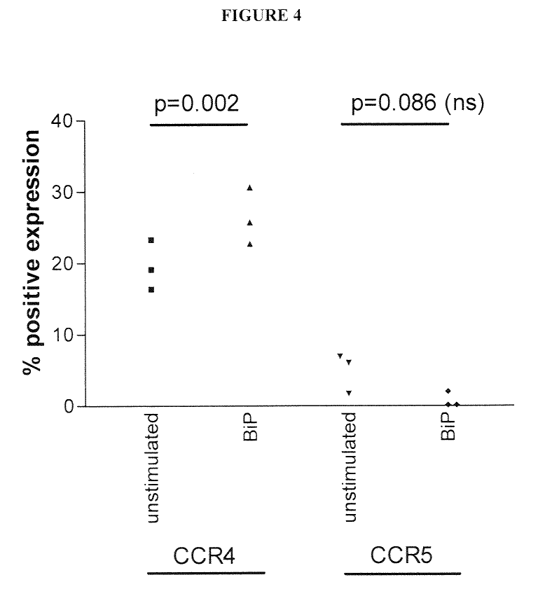

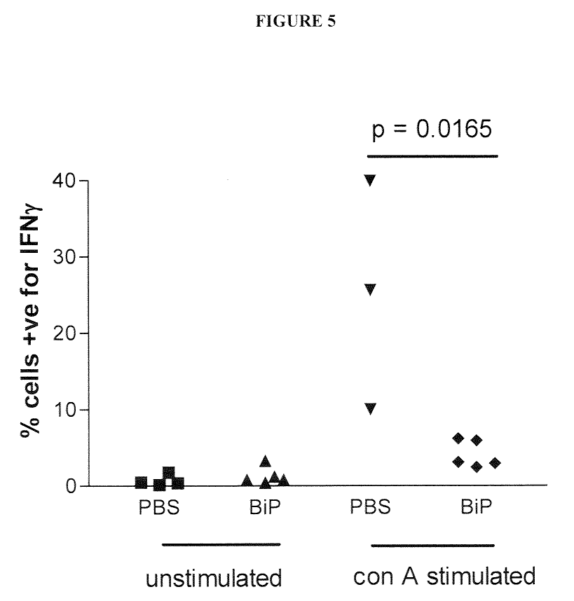

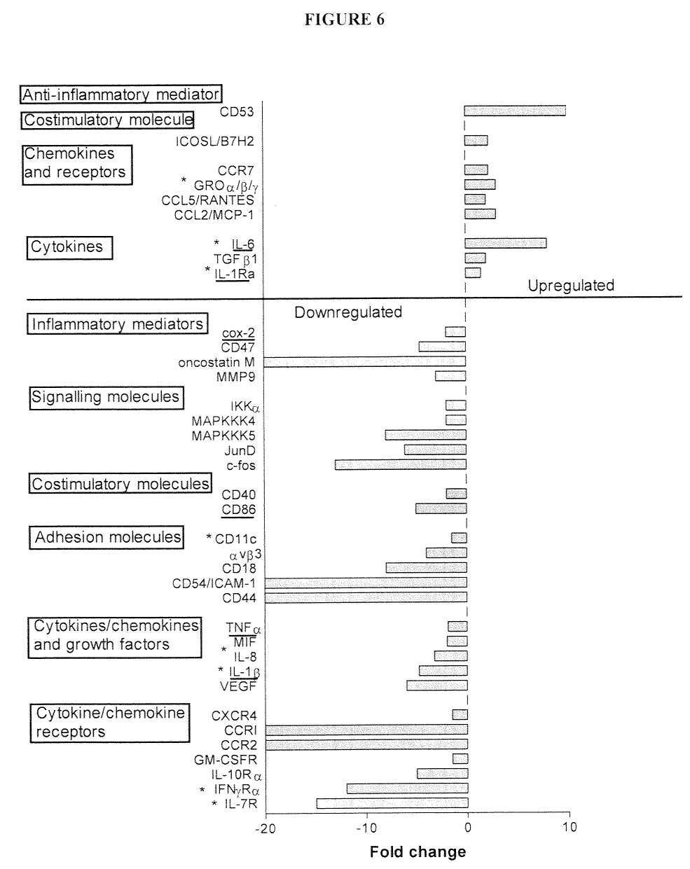

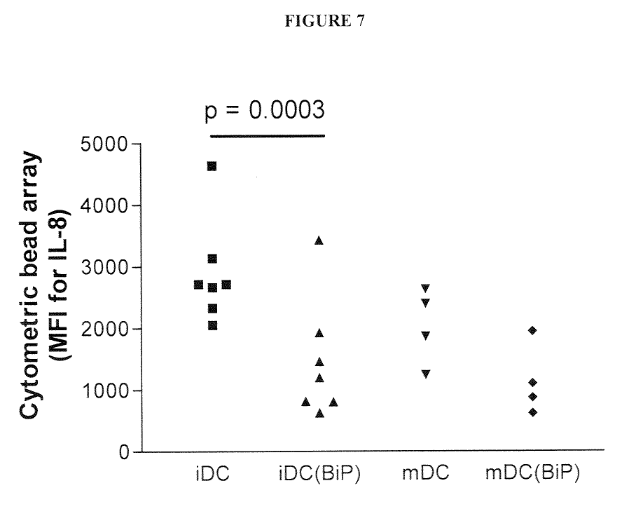

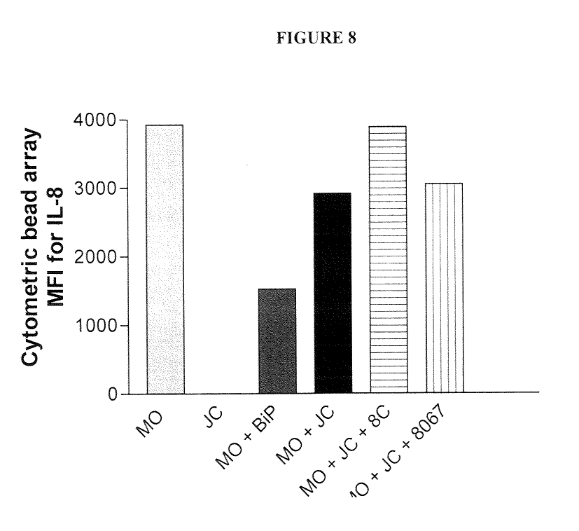

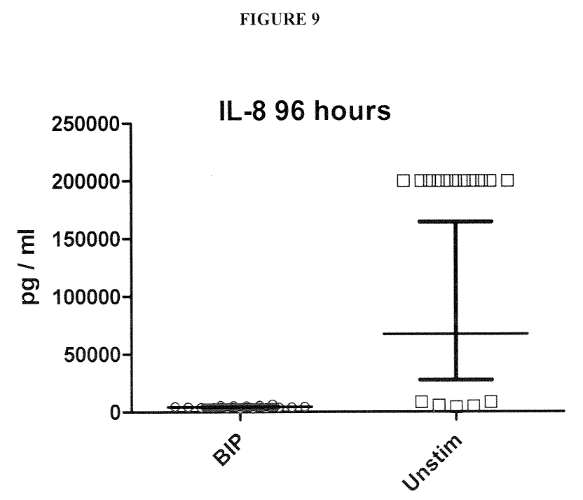

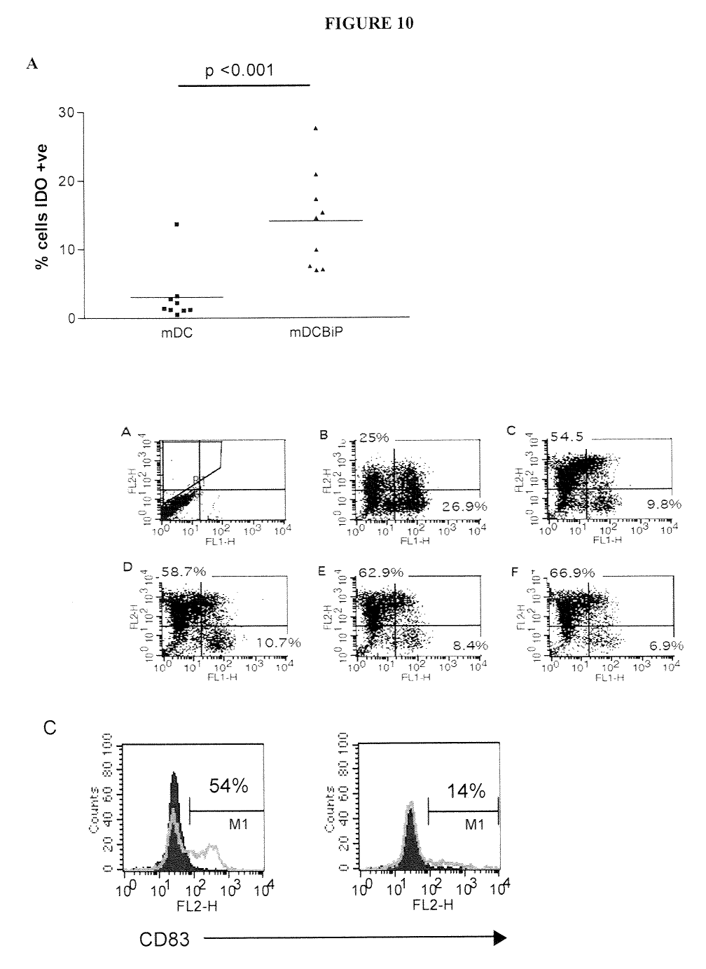

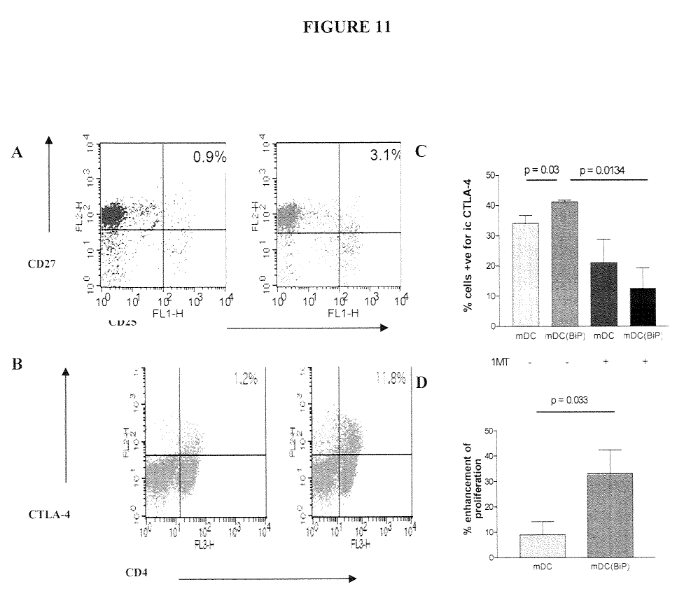

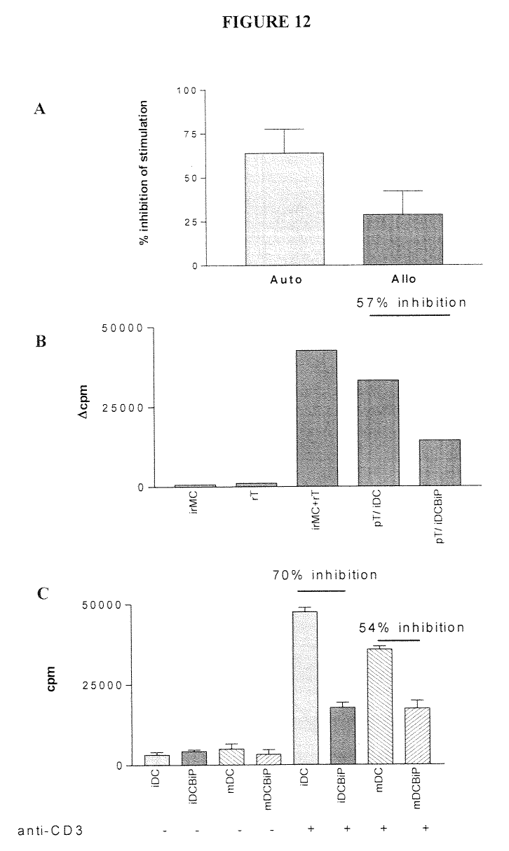

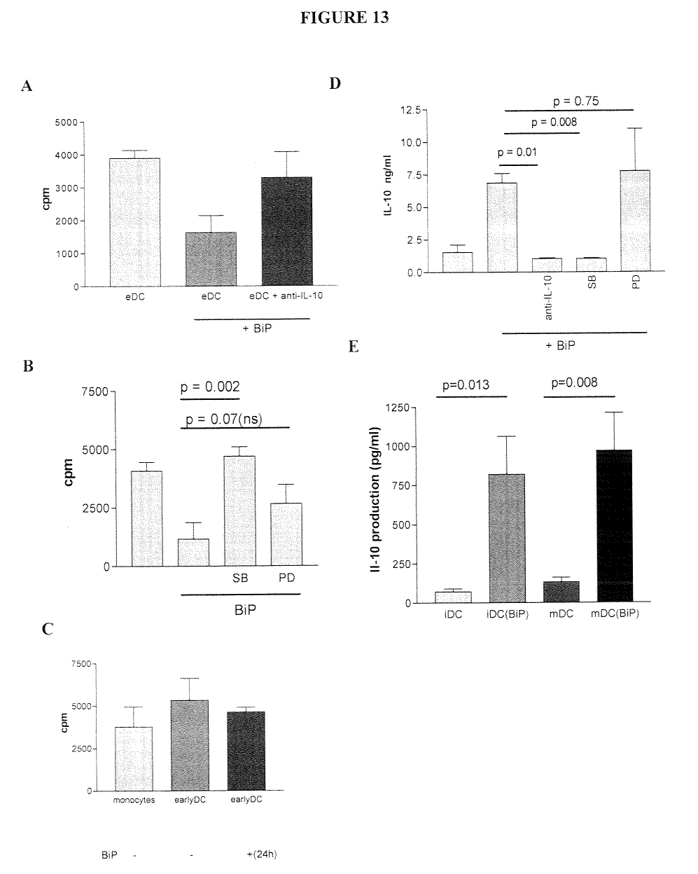

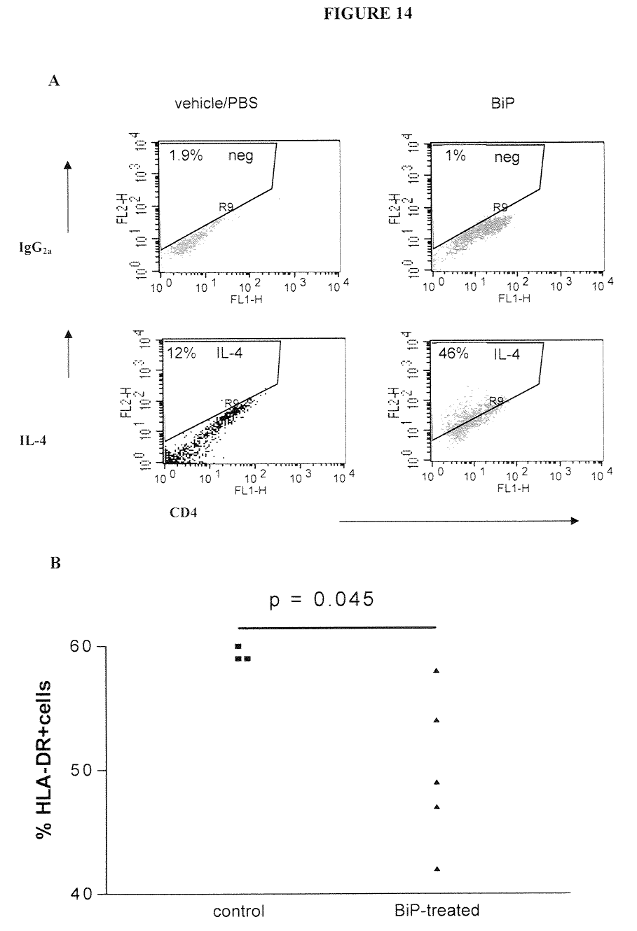

Moreover, we show that the IDO expression by the DC differentiated with BiP increases the intracellular CTLA-4 expression of T cells. This is another marker for regulatory T cells and has much to do with the decreased proliferative responses shown by these cells. FIGS. 3a,b,c herein (and also FIGS. 10 and/or 11) demonstrate this. Without wishing to be bound by theory, this may be the proof that DC directly effect the Tregs. Thus, IDO and/or CTLA-4 are preferred marker(s) of the invention.

The CCR4, CD8, CD122, 4, 25, 27, FoxP3 markers are all T cell markers specifically of regulatory T cells.

CD4CD25hi with either CD27hi and FoxP3 are the most commonly referred to Tregs but CD8 CD122 have also been defined (e.g. in mice). We disclose that the latter increases in humans in vitro and in mice ex vivo and in vivo. Thus these are particularly preferred markers of the invention.

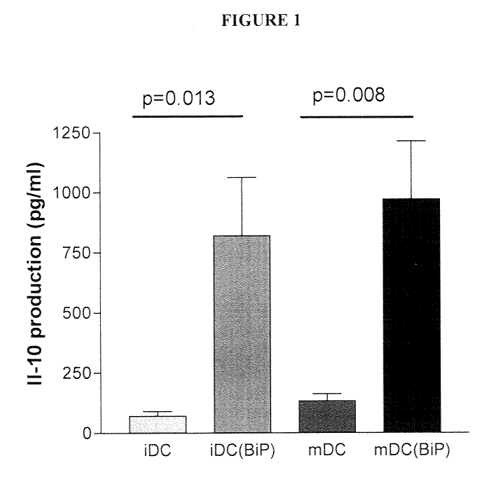

The cytokines are IL-10, IL-4, IL5, IL-6 and IFN. We show that in most in vitro systems where cytokines can accumulate BiP will cause the copious production of IL-10. In this case in the DC cultures there was also much IL-10 (FIG. 1). In vivo BiP caused increased IL-4 production by human cells grown interperitoneally in SCID mice (see examples) and it has also been shown that IFN is produced. IL-6 (see examples) is reduced in the transplants. Ex vivo in the mouse we disclose that BiP will stimulate production of IL-4, IL-5, IL-10 and IFNgamma compared with vehicle control which just produces IFN. Thus these cytokine groupings represent further preferred groupings of markers according to the present invention.

### Dendritic Cells

As is understood in the art, dendritic cells (DCs) are immune cells and form part of the mammalian immune system. Their main function is to process antigen material and present it on their surface to other cells of the immune system. Dendritic cells are present in small quantities in tissues that are in contact with the external environment, mainly the skin and the inner lining of the nose, lungs, stomach and intestines. They can also be found at an immature state in the blood. Once activated, they migrate to the lymphoid tissues where they interact with T cells and B cells to initiate and shape the immune response.

In vitro dendritic cells may be differentiated from peripheral blood mononuclear cells in the presence of cytokines—such as granulocyte-macrophage colony stimulating factor (GM-CSF) and interleukin (IL)-4. In vivo these cells have an important role in determining the appropriate immune response that follows antigenic stimulus. The maturation status, cell surface expressed antigens and cytokine production of the dendritic cells and the subsequent cross-talk between dendritic cells and T cells are all crucial in dictating whether TH1 cells drive a full inflammatory response or there is induction of tolerance via TH2 and/or regulatory T cells (Treg).

In one embodiment, the dendritic cell is a monocyte derived dendritic cell obtained or obtainable by culturing peripheral blood monocytes in the presence of one or more cytokines—such as GM-CSF and/or IL-4—for at least about 3 days or at least about 7 days, optionally in the presence of a maturation agent—such as lipopolysaccharide or tumor necrosis factor α.

As used herein an “early immature dendritic cell” refers to a monocyte derived dendritic cell obtained or obtainable by culturing peripheral blood monocytes in the presence of one or more cytokines—such as GM-CSF and IL-4—for at least about 3 days.

As used herein an “immature dendritic cell” refers to a monocyte derived dendritic cell obtained or obtainable by culturing peripheral blood monocytes in the presence of one or more cytokines—such as GM-CSF and/or IL-4—for at least about 7 days.

As used herein an “mature dendritic cell” refers to a monocyte derived dendritic cell obtained or obtainable by culturing peripheral blood monocyte in the presence of one or more cytokines—such as GM-CSF and/or IL-4—for at least about 7 days and also in the presence of a maturation agent—such as lipopolysaccharide or tumor necrosis factor α for the last 2 days.

In another embodiment, the mature dendritic cell may be a mature tolerogenic dendritic cell.

### Generating Regulatory T-Cells

In a further aspect, there is provided the use of BiP for generating regulatory T-cells in vivo or in vitro.

As described herein, the inventors have surprisingly found that BiP upregulates regulatory T-cells. T cells cultured together with dendritic cells induced with BiP become regulatory in function. They are capable of suppressing the responses of autologous T cells to anti-CD3 antibody.

T cells may be purified from peripheral blood mononuclear cells and cultured with allogeneic and/or autologous dendritic cells. Suitably, the dendritic cells are washed and irradiated, at a ratio of 10 T cells:1 dendritic cell. The cells are co-cultured for about 4 days and then removed.

T cells may be purified from peripheral blood mononuclear cells and cultured in the presence of BiP (eg. 20 μg/ml) for about 4 days.

Regulatory T cell activity may be measured using the methods described herein.

### Generating Tolorogenic Dendritic Cells

In a further aspect, there is provided the use of BiP for generating tolorogenic dendritic cells in vitro or in vivo.

Briefly, BIP is added to monocytes in the presence of cytokines—such as GM-CSF and IL-4, followed by maturation using, for example, lipopolysaccharide.

In one specific embodiment, tolerogenic dendritic cells are generated by adding 20 μg/ml BiP to monocytes with GM-CSF and IL-4. These cells are replenished 3 times over 6 or 7 days and maturation is achieved by adding lipopolysaccharide (eg. LPS 500 ng/ml lipopolysaccharide) for the last 2 days.

### Kits

The markers described herein may be detected using kits.

Such a kit may comprise containers, each with one or more of the various reagents (typically in concentrated form) utilised in the methods.

Suitably, the kit comprises a labelled (eg. fluorescently labelled) conjugated antibody (eg. monoclonal antibody) to the markers CD14, CD80, CD83, CD86, CD85j, HLA-DR, CD11, CD1a, CD4, CD8, CD25, CD27, CD122, CTLA-4, FoxP3, IL-10, IL-4 and IFNγ.

Suitably, the kit comprises a labelled (eg. fluorescently labelled) conjugated antibody (eg. monoclonal antibody) to the markers, CD80, CD 11, CD1a, CD4, CD8, CD25, CD27, CD122, CTLA-4, FoxP3, IL-10, IL-4 and IFNγ.

Fluorescent conjugated monoclonal antibodies to each of the required cell surface markers may include CD14.phycoerthrin (PE), CD80.PE, CD83.PE, CD86. (fluorescein isothiocyanate (FITC), CD85j.FITC, HLA-DR.FITC, CD11c.PE and/or CD1a.FITC.

Fluorescent conjugated monoclonal antibodies to each of the required cell surface markers may include CD80.PE, CD11c.PE and CD1a.FITC.

Fluorescent conjugated monoclonal antibodies to the intracellular marker IDO may include IDO.PE.

Fluorescent conjugated monoclonal antibodies for the T cell surface markers may include: CD4.PE, CD8.peridium chlorophyll (PerCP), CD25.FITC, CD27.PE, CD122.PE.

Fluorescent conjugated monoclonal antibodies to the intracellular markers CTLA-4 and FoxP3 for regulatory T cells may include CTLA-4.PE and FoxP3.PE.

Fluorescent conjugated monoclonal antibodies to the intracellular anti-cytokine antibodies may include anti-IL-10.PE, IL-4.FITC and IFNγ.PE for DC and T cell analysis.

The kit may also comprise one or more lectins—such as concanavalin A (eg. a stock solution of 1 mg/ml).

The kit may also comprise anti-CD3 and/or CD28 beads for functional tests.

The kit may also comprise immunofluorescent buffer (eg. 10× concentrate) and/or saponin solution (eg. 3% saponin solution).

A set of instructions will also typically be included.

### Antibodies

Antibodies may be used to detect one or more of the markers described herein.

In one embodiment, antibodies are described that specifically hybridise to CD14, CD80, CD83, CD86, CD85j, HLA-DR, CD11, CD1, CD4, CD8, CD25, CD27, CD122, CTLA-4, FoxP3, IL-10, IL-4, IL-5, IL-6, IFNγ, IDO, CCR-4 and/or CD40.

In one embodiment, antibodies are described that specifically hybridise to CD80, CD11, CD1, CD4, CD8, CD25, CD27, CD122, CTLA-4, FoxP3, IL-10, IL-4, IL-5, IL-6, IFNγ, IDO and/or CCR-4.

In one embodiment, a plurality of antibodies (eg. at least two) is used.

In one embodiment the invention relates to a kit for determining the functionality of BiP in vivo comprising a plurality of labelled antibodies, wherein said labelled antibodies are selected from labelled antibodies which specifically hybridise to one or more of CD14, CD80, CD83, CD86, CD85j, HLA-DR, CD11, CD1, CD4, CD8, CD25, CD27, CD122, CTLA-4, FoxP3, IL-10, IL-4, IL-5, IL-6, IFNγ, IDO, CCR-4 and CD40. Suitably said labelled antibodies are selected from labelled antibodies which specifically hybridise to one or more of CD80, CD11, CD1, CD4, CD8, CD25, CD27, CD122, CTLA-4, FoxP3, IL-10, IL-4, IL-5, IL-6, IFNγ, IDO and CCR-4.

Suitably three or more antibodies are present, suitably four or more antibodies are present, suitably five or more antibodies are present, suitably six or more antibodies are present, suitably eight or more antibodies are present, suitably ten or more antibodies are present, suitably 12 or more antibodies are present, suitably 15 or more antibodies are present, suitably 17 or more antibodies are present.

In one embodiment, the antibody is labelled (eg. fluorescently labelled).

In one embodiment, the antibody is a monoclonal antibody.

Monoclonal antibodies may be purchased or they may be readily produced by one skilled in the art. The general methodology for making monoclonal antibodies by hybridomas is well known. Immortal antibody-producing cell lines may be created by cell fusion, and also by other techniques such as direct transformation of B lymphocytes with oncogenic DNA, or transfection with Epstein-Barr virus. Monoclonal antibodies also may be prepared using any technique, which provides for the production of antibody molecules by continuous cell lines in culture. These include, but are not limited to, the hybridoma technique originally described by Koehler and Milstein (1975 Nature 256:495-497), the human B-cell hybridoma technique (Kosbor et al (1983) Immunol Today 4:72; Cote et al (1983) Proc Natl Acad Sci 80:2026-2030) and the EBV-hybridoma technique (Cole et al (1985) Monoclonal Antibodies and Cancer Therapy, Alan R Liss Inc, pp 77-96).

### Fragments/Variants/Homologues/Derivatives

The present invention encompasses the use of fragments, variants, homologues, and derivatives of BiP.

The term “variant” is used to mean a naturally occurring polypeptide or nucleotide sequences which differs from a wild-type sequence.

The term “fragment” indicates that a polypeptide or nucleotide sequence comprises a fraction of a wild-type sequence. It may comprise one or more large contiguous sections of sequence or a plurality of small sections. The sequence may also comprise other elements of sequence, for example, it may be a fusion protein with another protein. Preferably the sequence comprises at least 50%, more preferably at least 65%, more preferably at least 80%, most preferably at least 90% of the wild-type sequence.

The term “homologue” means an entity having a certain homology with the subject amino acid sequences and the subject nucleotide sequences. Here, the term “homology” can be equated with “identity”.

In the present context, a homologous sequence is taken to include an amino acid sequence, which may be at least 75, 85 or 90% identical, preferably at least 95, 96, 97 or 98% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence, which may be at least 75, 85 or 90% identical, preferably at least 95, 96, 97 or 98% identical to the subject sequence. Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons may be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalizing unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix—such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues, which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example, according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) may occur i.e. like-for-like substitution—such as basic for basic, acidic for acidic, polar for polar etc. Non-homologous substitution may also occur i.e. from one class of residue to another or alternatively involving the inclusion of unnatural amino acids—such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids—such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#}, L-norleucine*, L-norvaline*, p-nitro-L-phenylalanine*, L-hydroxyprolie^{#}, L-thioproline*, methyl derivatives of phenylalanine (Phe)—such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups—such as methyl, ethyl or propyl groups—in addition to amino acid spacers—such as glycine or β-alanine residues. A further form of variation involves the presence of one or more amino acid residues in peptoid form will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example, Simon R J et al., PNAS (1992) 89(20), 9367-9371 and Horwell D C, Trends Biotechnol. (1995) 13(4), 132-134.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences may be modified by any method available in the art. Such modifications may be carried out to enhance the in vivo activity or life span of nucleotide sequences useful in the present invention.

Preferably, the fragment is a functional fragment. As used herein, the term “functional fragment” refers to a fragment of BiP which is capable of eliciting at least part of an activity of the full length BiP protein. In particular, the functional fragment may have one or more of the following functions: (i) causes CD14+ cells to release IL-10; (ii) stimulates CD8+ cells to proliferate and release IL-10; (iii) inhibits the recall antigen response; (iv) activates the expression of an array of anti-inflammatory genes in monocytes, including the migration inhibitory factor (MIF), the soluble TNF receptor II and TIMPs; (v) inhibits oestoclast maturation; and/or (vi) inhibits bone resorption.

Preferably, the functional fragment is at least 20 amino acids, more preferably at least 50 amino acids and most preferably at least 100 amino acids in length. Particularly preferred fragments comprise a conserved region which has been found to be homologous to a number of naturally occurring BiP proteins. Such conserved regions are considered to have a specific function.

The term “functional homologue” as used herein refers to a homologue that retains at least part of an activity of the full length BiP protein. In particular, it is preferred that the functional homologue has at least one of the following functions: (i) causes CD14+ cells to release IL-10; (ii) stimulates CD8+ cells to proliferate and release IL-10; (iii) inhibits the recall antigen response; (iv) activates the expression of an array of anti-inflammatory genes in monocytes, including the migration inhibitory factor (MIF), the soluble TNF receptor II and TIMPs; (v) inhibits oestoclast maturation; and/or (vi) inhibits bone resorption.

### General Recombinant DNA Methodology Techniques

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N. Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

### Further Applications

The methods and uses of the invention are suitably in vitro methods or uses. In this embodiment suitably the sample is handled/analysed in vitro, although of course such a sample will typically have been collected from a subject.

In some embodiments the methods and uses of the invention are suitably in vivo methods or uses (or ex-vivo methods or uses).

The invention will now be further described by way of Examples, which are meant to serve to assist one of ordinary skill in the art in carrying out the invention and are not intended in any way to limit the scope of the invention.

### EXAMPLES

### Materials & Methods

### Isolation of PBMC, T Cells and MO

Peripheral blood mononuclear cells (PBMC) were isolated, by density centrifugation on Lymphoprep, from heparinised venous blood from healthy volunteers, after informed consent and approval of the project by the Guy's and St Thomas' Hospital Ethical Committee, by density centrifugation over Lymphoprep (Nycomed-Pharma, Amersham, UK). T cells and MO were purified from PBMC by negative selection using the appropriate immunomagnetic kit (Dynal, Bromborough, UK).

### Differentiation of Monocyte-Derived iDC and mDC

Purified monocytes (>85% monocytes)(1.5.10⁶/flask)(Corning Costar, High Wycombe, UK) were incubated in 5 ml of tissue culture medium (TCM)(RPMI 1640 medium (Sigma, Poole, UK) supplemented with 10% heat-inactivated foetal calf serum (FCS)(Life Technologies, Paisley, UK). iDC were generated using granulocyte macrophage—colony stimulating factor (GM-CSF) (1000 U/ml)(Novartis Research Institute, Vienna, Austria) and IL-4 (500 U/ml) (R&D Systems, Oxford, UK). Monocytes were cultured for 3 or 7 days with cytokines, either alone or in the presence of rhuBiP (20 μg/ml). Mature DC were generated by addition of 500 ng/ml lipopolysaccharide (LPS) for the final 2 days of culture.

### Immunofluorescent Staining and Flow Cytometric Analysis

Immunofluorescent staining was performed as follows when required. Cells were suspended in immunofluorescent buffer (IF) (PBS/0.1% BSA/0.05% azide) washed and incubated for 10 mins on ice with normal human serum. After washing twice in IF buffer, saturating concentrations of fluorescein isothiocyanate (FITC)-, phycoerythrin (PE) or peridium chlorophyll (Per-CP)-conjugated monoclonal antibodies (mAb) recognizing human CD14, CD86, CD80, HLA-DR, CD11c, CD1a, CD83, CD40 and CD85j, (all from Becton Dickinson/PharMingen, Oxford, UK) were added singly or in combination, as stated in the text, to cells for 20 mins on ice. Cells were then washed twice in IF buffer and analysed. Isotype controls, IgG1 and IgG2a (Becton Dickinson, Oxford, UK) were used in parallel.

Intracellular staining was carried out with fixed cells incubated with 1% paraformaldehyde in IF buffer for 5 minutes at room temperature. The cells were washed twice with IF buffer containing 0.3% saponin for permeabilization. 0.3% saponin was then present in all IF buffer used thereafter. CTLA-4/CD152 (BD), FoxP3 (eBioscience) and indolamine 2,3-dioxygenase (Santa Cruz) and cytokines required intracellular staining. Antibodies were added at the pre-determined optimal concentration for 20 mins and then the cells were washed twice with IF buffer and analysed. Isotype controls, IgG1 and IgG2a (Becton Dickinson, Oxford, UK) were used in parallel. Cells were analysed on a FACScan cytometer using Cellquest software (Becton Dickinson, Oxford, UK).

### Quantification of Cytokines

Supernatant samples were collected on day 1, 3 and 7 for iDC and from the final cultures of the T cells taken from the iDC/mDC-T cell allogeneic test system and set up with autologous cells to determine if there was any regulatory activity. The samples were aliquoted and frozen at −70° C. until required. Production IL-10 was quantified by ELISA using paired antibodies and recombinant standards (PharMingen, Oxford, UK) according to the recommendations of the manufacturer. In addition some were analysed by cytometric bead assay (BD, Oxford, UK) according to the manufacturers instructions.

### Generation of Regulatory T Cells

T cells were purified from peripheral blood mononuclear cells and set up in culture with either allogeneic or autologous dendritic cells, which had been washed and irradiated, at a ratio of 10 T cells:1 DC. The cells were co-cultured in a 24 well plate in 1 ml/well, in RPMI 1640 supplemented with 10% foetal calf serum, for 4 days then the T cells were removed and washed before being investigated for the presence of T regulatory cells (see below)

### Determination of Regulatory T Cell Activity

Purified T cells (>95% T cells) (2.10⁶/well) either autologous or allogeneic were cultured in the absence or presence of irradiated DC populations in 24-well flat-bottomed plates at a IDC: 10 T cell ratio for 4 days. T cells were isolated from the iDC/mDC allogeneic cultures and incubated with anti-HLA-DR antibody (clone L243, ATCC, Rockville USA, 1/20 dilution) and the DC removed with goat anti-mouse coated immunomagnetic beads (Dynal, Bromborough, UK). T cells (10⁶/ml; 96 well plate) were then set up in an autologous culture with fresh T cells and irradiated PBMC (1:1:1 cell ratio) and cultured with or without anti-CD3 (clone OKT3, ATCC, Rockville, USA, 1/2000 dilution) for three days. The cells were pulsed with [³H] thymidine (Tdr)(0.2 μCi/well) (Pharmacia Biotech, Amersham, UK) for the final 18 h of culture.

### Murine In Vivo Experiments

The biomarkers were investigated in two situations: 1) when 50.10⁶ human peripheral blood mononuclear cells were injected intraperitoneally, with or without BiP (100 μg), into a mouse and the cells were harvested from the peritoneum 11 days later; or (2) BiP (200 μg) was injected intravenously into a mouse and the spleens and lymph nodes removed 11 days later.

### Isolation of Mouse Splenocytes and Lymph Node Cells

Mice were sacrificed at the end of the experiment and the spleens and lymph nodes removed. The spleens and lymph nodes were then gently pushed through a fine mesh to produce a single cell suspension. The cells were then either set up in culture or stained as for human cells following the protocol below.

IF staining of mouse cells was as described for human cells above.

### Example 1

### BiP Delays/Inhibits the Development of Dendritic Cells Derived from Human Peripheral Blood Monocytes

The intricacies of the interactions between the innate and adaptive immune system that determine the type of immune response that follows a particular antigenic assault are currently being dissected. Undoubtedly, one of the most important players in the initiation of an appropriate immune response is the dendritic cell (DC). To achieve this, the maturation status of the DC, its local environment and DC production of inflammatory mediators are important elements of the response, and the subsequent cross-talk between DC and T cells is crucial in dictating whether TH1 cells drive a full inflammatory response or there is induction of tolerance via TH2 and/or regulatory T cells (Treg).

The affect of BiP on monocyte-derived DC, in the presence of GM-CSF and IL-4, with and without lipopolysaccharide to mature the DC, caused the changes listed in Table 1.

These changes appear to be time dependent in some cases (eg. for CD40, CD86, CD80, CD85j and CD14). There is also a difference in the MFI which indicates that there may be fewer molecules/cell. CD83 is the mDC marker and the consistently low level of expression in mDC(BiP) shows full maturation is not achieved.

In addition, BiP-treated DC produce significantly more IL-10 than untreated DC, as shown in FIG. 1.

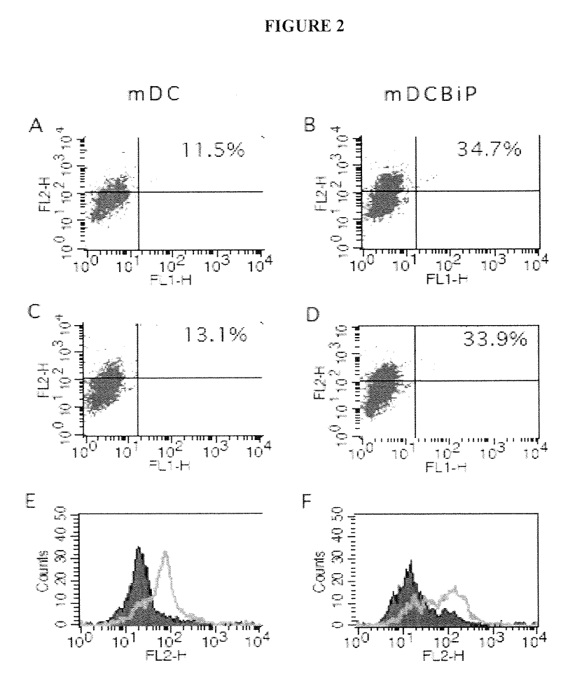

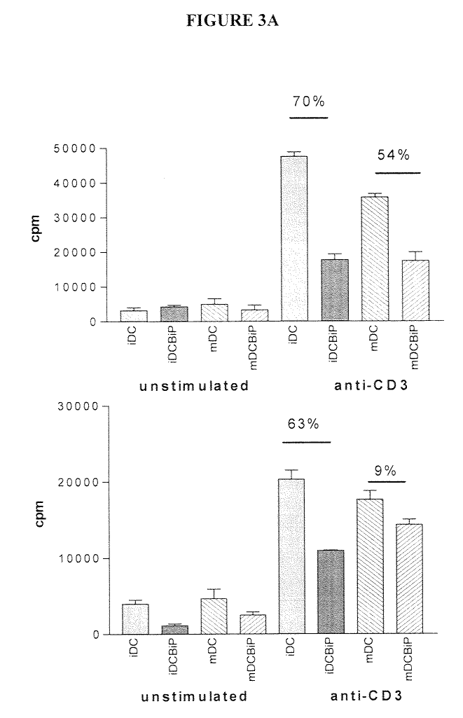

The numbers of cells containing intracellular indoleamine 2,3-dioxygenase (IDO), an enzyme involved in tryptophan catabolism, is also raised several fold in BiP stimulated mDC (8.5±7 fold increase (range 2.0-20 fold) (mDC, 4.3±6.2% versus mDC(BiP), 17.6±8.6%, n=4, p=0.047), as shown in FIG. 2.

IDO is important in tolerogenic DC and up until now very few molecules have been shown to upregulate IDO without extensive manipulation in vitro.

Also raised is the expression of CD85j or immunoglobulin-like transcript (ILT)₂, an inhibitory molecule that binds SHP1 and precipitates inhibition of T cell reactivity. In experiments with matured DC there is a significant rise in the expression of this molecule (mDC, 72.5±6.4% versus mDC(BiP), 87.2±3.5%; p=0.008, n=4).

### Example 2

### Human T Regulatory Cells

There are several different types of regulatory cells described in the literature but by far the most reported are the CD4CD25^{hi}CD27^{hi}/CD4CD25^{hi}FoxP3 cells. We have investigated whether BiP upregulates these cells, or other regulatory T cell subpopulations—such as Tr1 and CD8CD122+ cells. We have studied the development of regulatory T cells in two different ways: 1) following co-culture with BiP treated DC, after the latter have been washed free of BiP; and 2) whether BiP has the ability to act directly on the small number of T cells that we know have a BiP receptor expressed on their cell surface.

Our investigations have come to the conclusion that T cells that are cultured together with DC(BiP) become regulatory in function. They are capable of suppressing the responses of autologous T cells to anti-CD3 antibody, as shown in FIGS. 3a, 3b and 3c)

Also significantly raised after contact with BiP treated DC was intracellular CTLA-4/CD152 (DC, 25±12 versus DC_{(BiP)}, 31±12, p=0.04, n=4).

The analysis of the cell surface and intracellular markers for T suppressor cells also showed a marked upregulation of cell surface CCR4 (a TH2 marker) both after contact with BiP treated DC ((mDC, 10.8±0.8% versus mDC_{(BiP)}17.3±2.0%, p=0.024; n=4) and also when T cells are stimulated with BiP directly.

### Example 3

### The Direct Effect of BiP on T Cells

The CCR4 marker is a TH2 marker and CCR5 is a TH1 marker. The inverse changes shown in FIG. 4 indicate a skewing of the cell population from TH1 towards TH2.

Another group of regulatory T cells recently described are characterised by the expression of CD8CD122. These cells show significant increases in expression after 7 days incubation with BiP (T cells alone, 32.7%±6.6% versus BiP treated T cells, 75.6%±23.5%; p=0.039, n=3).

In experiments where purified T cells or PBMC were treated directly with BiP for 4 or 7/8 days we observed that when put into the same regulatory T cell assay with fresh T cells and irradiated PBMC (1:1:1) there was considerable inhibition of proliferation. The control cultures, either in the presence of human serum albumin (HSA) or in tissue culture medium (TCM) alone show a level of inhibition up to 15% but the BiP treated cultures showed at least double this and it could not be reversed by the addition of anti-IL-10. The data are shown in Table 2.

### Example 4

### Human Cells Injected Intra-Peritoneally into SCID Mice

This is a method of investigating the effect of an agent, such as BiP, on human cells in a ‘living test-tube’. These mice lack T and B cells and therefore do not raise an immune response to the injected cells.

In each of three experiments, two/three mice were injected either with BiP treated human peripheral blood mononuclear cells (hPBMC) (50.10⁶/mouse) and or untreated hPBMC. The mice were sacrificed 11 days later, the peritoneal cavity washed out and the cells collected for immunofluorescence and flow cytometric analysis. The cells were particularly stained for TH2 markers and intracellular anti-inflammatory cytokines.

Mice showed increased numbers of cells with the following intracellular cytokines:

IL-4 PBS, 8% versus BiP treated, 16.2±0.5%

IL-5 PBS, 15% versus BiP treated, 29±7%

IFNγ PBS, 38% versus BiP treated, 23.5±4%

These changes are indicative of a skewing towards the development of TH2 cell populations. IFN gamma is a TH1 cytokine and IL-4 and IL-5 are TH2.

### Example 5

### Intravenous Injection of BiP (200 μg/Mouse) into DBA Mice

Mice were given a single dose of BiP and left for 11 days before they were sacrificed and their spleens and lymph nodes removed for analysis. The splenocytes and lymph node cells were pooled and used in immunofluorescence and flow cytometry studies. They were also set up in a simple recall antigen proliferation study to check that BiP had been successfully administered at time-point zero.

Cells were examined for: CD3, CD4, CD8, CD25, CD122, CCR4, intracellular CTLA-4 and cells actively producing IL-10 and interferon (IFN)γ.

Results show:

Increased % of CD8CD122+ cells (untreated, 20.9±2.8% versus BiP treated, 26.3±3.7%; n=3); and

Increased intracellular CTLA-4.

The decrease in the number of cells producing IFN gamma after being stimulated by concanavalin A, a stimulant of TH1 cells (untreated, 28.8±6.7% versus BiP treated, 3.9±1.7%, n=3 p=0.013) was particularly marked. There was also a higher number of IL-10 producing cells in the BiP treated mice when these cells were isolated (untreated, 2.6±1.5% versus BiP treated, 6.1±1.3%, n=3, p=0.041). This may be particularly important because it shows that BiP prevents a conventional response to a

TH1 stimulant.

TABLE 1
Surface expression of phenotypic markers
a)

| | % cells | % cells | |
|---|---|---|---|
| | Early | Early | |
| Marker | iDC | IDC(BiP) | |
| CD14 | 54 ± 28 | 91 ± 6 | |
| CD83 | 9 ± 7 | 2. ± 2 | |
| CD1a+ | 15 ± 12 | 1 ± 2 | p = 0.06 ns |
| CD11c+ | 88 ± 7 | 81 ± 7 | |
| CD86+ | 15 ± 4 | 3 ± 1 | p = 0.02 |
| CD80+ | 70 ± 5 | 36 ± 15 | p = 0.008 |
| HLA-DR | 90 ± 6 | 31 ± 7 | p = 0.001 |

b)

| | | iDC | | | mDC | |
|---|---|---|---|---|---|---|
| | iDC | (BiP) | | mDC | (BiP) | |
| CD14+ | 61 ± 14 | 59 ± 22 | | 18 ± 22 | 57 ± 22 | p = |
| | | | | | | 0.06 |
| | | | | | | ns |
| CD83+ | 7 ± 4 | 10 ± 5 | | 39 ± 15 | 10 ± 4 | p = |
| | | | | | | 0.006 |
| CD1a+ | 87 ± 7 | 83 ± 9 | | 83 ± 7 | 76 ± 14 | |
| CD11c+ | 97 ± 2 | 97 ± 2 | | 90 ± 15 | 94 ± 5 | |
| CD86+ | 28 ± 13 | 31 ± 11 | | 60 ± 14 | 26 ± 9 | p = |
| | | | | | | 0.045 |
| CD80+ | 91 ± 5 | 88 ± 7 | | 94 ± 4 | 91 ± 6 | |
| HLA-DR+ | 98 ± 2 | 96 ± 2 | | 96 ± 3 | 96 ± 1 | |
| CD40+ | 12 ± 11 | 28 ± 20 | p = | 36 ± 18 | 32 ± 24 | |
| | | | 0.02 | | | |
| CD40+85j+ | 10 ± 9 | 26 ± 19 | p = | 29 ± 13 | 29 ± 23 | |
| | | | 0.017 | | | |
| CD210+ | 13 ± 15 | 12 ± 9 | | 23 ± 17 | 19 ± 17 | |

c)

| | MFI | MFI | |
|---|---|---|---|
| | mDC | mDC(BiP) | |
| CD14 | 60 ± 19 | 366 ± 216 | p = 0.04 |
| CD86 | 343 ± 49 | 112 ± 14 | p = 0.04 |
| CD80 | 946 ± 166 | 395 ± 31 | p = 0.07 ns |
| CD1a | 1219 ± 400 | 685 ± 145 | p = 0.0 |
| HLA-DR | 2843 ± 168 | 1660 ± 167 | p = 0.044 |
| | MFI | MFI | |
| | iDC | iDC(BiP) | |
| CD11c | 1511 ± 262 | 2246 ± 329 | p = 0.026 |

Purified monocytes were cultured with and without BiP for three days (early DC/early DC(BiP)) or 7 days with GM-CSF and IL-4 (iDC/iDC(BiP)) or for 7 days with additional LPS for the final 2 days (mDC/mDC(BiP)). *BiP [20 μg/ml] was added at the same time as the cytokines. (a) and (b): Results are expressed as mean and standard error (mean ±SE) of the % positive cells from 4 or 7 different experiments, respectively. (c) Results show the mean ±SE of the mean fluorescence intensity (MFI) of up to 7 experiments.

TABLE 2

| | | % change | % change |
|---|---|---|---|
| | % change | Anti-CD3 + | Anti-CD3 |
| | Anti-CD3 | anti-IL-10 | no contact |
| 1 T(HSA) | ↓15 | ↓11 | ↑35 |
| T(BiP) | ↓34 | ↓32 | ↑27 |
| 3 T(TCM) | ↓14 | ↓0 | |
| T(BiP) | ↓46 | ↓34 | |

### Example 6

We demonstrate that if cells from mice given BiP 11 days previously are harvested and then cultured overnight with con A (mitogen) there is a significant reduction in IFN production by the BiP treated mouse cells. This is shown in FIG. 5. This shows good anti-inflammatory function. Thus reduction of IFN (decreased IFN production) is an excellent marker of BiP function in vivo.

### Example 7

### BiP Downregulates IL-8 Production

Affymetrix™ gene array performed by Leicester University under contract showed that monocytes cultured with BiP for 24 h compared with unstimulated monocytes had downregulated the IL-8 gene. This was confirmed in two Affymetrix™ gene arrays and also in an inflammatory cytokine gene array.

FIG. 6 shows details of genes affected by BiP. In more detail, FIG. 6 shows analysis of BiP treated monocytes compared with resting monocytes, by Affymetrix™ gene array. The transcription profiles of the BiP stimulated and unstimulated MO were generated using the Human GeneChip U95Av2 which simultaneously measures 12500 gene transcripts. To identify genes differentially expressed in the presence of BiP, comparisons were made between the transcription profiles obtained from the MO using the analysis software dChip version 1.2 (32). Transcripts that showed an increase/decrease of at least 2 fold, and a difference in signal intensity between the baseline and experimental arrays of at least 100 units of fluorescence were selected for further evaluation.

In a series of experiments IL-8 was detected using cytometric bead array (Becton Dickinson) where the mean fluorescence intensity (MFI) is proportional to the concentration of cytokine. IL-8 was seen to be downregulated in culture supernatants from monocyte derived dendritic cells (DC) when cultured with BiP (20 μg/ml) in the presence of granulocyte-macrophage colony stimulating factor and IL-4 and in the presence (mDC) and absence (iDC) of lipopolysaccharide for the last 48 h of the 7d culture to mature the DC. FIG. 7 shows the data. In more detail, FIG. 7 shows IL-8 production by dendritic cells: Mean fluorescent intensity (MFI) from a cytometric bead array was indicative of the production of IL-8 by monocyte derived immature dendritic cells (iDC) or mature dendritic cells (mDC). BiP suppressed IL-8 production in both cases.

In a further experiment where Jurkat cells were used to look at the effect of cell surface expressed mammalian BiP on human monocytes, the high production of IL-8 by the monocyte culture was suppressed by addition of Jurkat cells (JC), detected by reduction of IL-8 MFI. Addition of anti-BiP monoclonal antibody (8C) to the co-culture restored the production of IL-8 to that of the monocyte culture alone (FIG. 8). In more detail, FIG. 8 shows that monocyte interaction with cell surface expressed BiP can reduce production of IL-8. Monocytes (MO) were cultured alone or with recombinant human BiP or BiP expressing Jurkat cells (JC). Addition of neutralizing anti-BiP antibody blocked the BiP interaction between JC and MO and returned the production to that of MO control while addition of an irrelevant isotype control did not affect production.

### Example 8

### Downregulation of IL-8 by BiP

Peripheral blood mononuclear cells (PBMC) were separated from heparinised blood taken from patients with rheumatoid arthritis being treated with methotrexate. PBMC were either left unstimulated or stimulated with BiP (20 μg/ml). Supernatants were taken at 96 h, aliquoted and stored at −80° C. until required. The cytokine concentration was estimated by MSD (Meso Scale Discovery) ELISA system The results are shown in FIG. 9.

In more detail, FIG. 9 shows downregulation of IL-8 by BiP. Supernatants from unstimulated or BiP stimulated cultures were analysed in a multicytokine array (MSD). The results from 16 paired samples are shown as median and 95 percent confidence limits. The BiP stimulated group produced significantly less IL-8 (p=0.0001).

### Example 10

BiP induces anti-inflammatory dendritic cells and regulatory T cells and downregulates rheumatoid synovial membrane inflammation. BiP suppresses rheumatoid synovitis.

Non-standard abbreviations: BiP, binding immunoglobulin protein; IDO, indoleamine 2,3 dioxygenase; SP, stress protein; HSP, heat shock protein; RASM, rheumatoid arthritis synovial membrane; CIA, collagen induced arthritis, MFI mean fluorescent intensity

Previously, BiP has been shown to have immunomodulatory functions. Herein we demonstrate that BiP will alter the differentiation of peripheral blood monocyte-derived dendritic cells (DC). BiP-treated DC express intracellular indoleamine 2,3-dioxygenase and show reduced surface expression of HLA-DR and CD86, increased CD85j and CD14 and produce copious interleukin (IL)-10. Furthermore, T cells co-cultured with BiP-treated DC develop regulatory function. These T cell populations had increased surface expression of CD4+CD25hiCD27hi and high levels of intracellular CTLA-4. Early addition of neutralizing anti-IL-10 antibody or the specific MAPK p38 inhibitor, SB203580 reversed the inhibition of DC differentiation by BiP. In vitro defined phenotypic and functional changes caused by BiP have been confirmed using in vivo models. BiP treated human peripheral blood mononuclear cells incubated in the peritoneal cavity of severe combined immunodeficient mice (SCID) show increased intracellular IL-4 and decreased HLA-DR expression. Similarly, administration of BiP to SCID mice transplanted with rheumatoid synovial membrane decreased cell infiltrate and downregulated CD86 and HLA-DR expression with concurrent reduction of human tumour necrosis factor α and IL-6 production. In conclusion, BiP is a powerful immunomodulator that can arrest inflammation through induction of tolerizing DC and the generation of T regulatory cells.

### Overview

The interactions between the innate and adaptive immune system that determine the type of immune response that follows a particular antigenic assault are currently being dissected. Probably one of the most important players in the initiation of an appropriate immune response is the dendritic cell (DC). The maturation status of the DC and its local environment, with respect to the presence of inflammatory mediators, influence the subsequent cross-talk between DC and T cells and are crucial factors in dictating whether T cells develop into T helper (Th)1(1) associated with the presence of interleukin (IL)-12, or Th17(2) cells, in the presence of tumour growth factor (TGF)β and IL-6, driving a full inflammatory response, or Th2, classically associated with the presence of IL-4, thus downregulating immune pathways (3). Production of IL-10 by DC in the early stages of culture and DC phenotype clearly also have an affect on the differentiation of regulatory T cells (4). The nature of the initiating molecules that drive a DC to become pro- or anti-inflammatory, however, is still uncertain.

Stress proteins (SP) or heat shock proteins (HSP) are evolutionarily highly conserved proteins (5). They have important intracellular functions as chaperones and protectors of cells from stress (5). More recently though they have increasingly been recognised as having extracellular functions by signalling through cell surface expressed receptors. Thus they are able to influence monocyte differentiation through ligation of surface expressed receptors, such as the toll-like receptors (TLR)₂ (6) and TLR4(7), CD40(8), LOX-1(9) and CD91(10; 11). The majority of these SP, such as HSP70, have been shown to stimulate monocytes to drive an overall pro-inflammatory response, reflecting the ‘danger’ hypothesis (12), with acceleration of full DC maturation and stimulation of interleukin (IL)-12, tumour necrosis factor (TNF)α and IL-1β production (6; 13). In direct contrast, we have shown that BiP, although a member of the HSP70 family, affects peripheral blood monocytes (14), via an as yet uncharacterised receptor, to reduce HLA-DR and CD86 expression combined with substantial and prolonged production of IL-10(14). A functional consequence of these changes was the reduced proliferation of human peripheral blood mononuclear cells (PBMC) to the recall antigen tuberculin PPD. In addition, BiP will drive differentiation of T cells towards a Th2 profile with anti-inflammatory properties. Thus we have expanded, from PBMC, BiP specific CD8 and CD4 T cell clones that produced IL-10, IL-4 and IL-5 in various combinations with little or no IFNγ (15). Similarly, in vitro investigation of the lymph node and spleen cells from mice given BiP secreted IL-4(16) and IL-10 on re-stimulation with BiP. In vivo, a single intravenous injection of BiP given to DBA/1 or HLA-DR1 transgenic mice prevented or treated ongoing collagen-induced arthritis (CIA). Importantly, splenocytes and lymph node cells from the BiP-treated animals given BiP intravenously, when adoptively transferred will prevent or treat CIA but this protective effect is ablated in IL-4 knockout mice (16). Thus there seems to be an analogy between the mouse and the human in the terms of BiP being able to stimulate the release of IL-4.

Collectively these results have led us to hypothesize that BiP is able to modify the immune response through interactions with monocytes and produce T cells that may have anti-inflammatory and immunomodulatory properties. In particular we have hypothesized that BiP may act after the initial inflammatory immune response to resolve the inflammation and restore homeostasis (17). In this study we present evidence that in vitro BiP stimulation modulates human monocyte differentiation into immature DC (iDC), inhibits development of mature DC (mDC) and that subsequent T cell contact with BiP-treated DC, either autologous or allogeneic, enhances regulatory T cell development. Validation of these in vitro immunomodulatory properties of BiP is shown in two xenogeneic in vivo models. Firstly, BiP stimulated the production of IL-4 from human PBMC incubated in the peritoneum of SCID mice. Secondly, BiP ablated the inflammatory cell infiltrate and suppressed TNFα and IL-6 production in small pieces of human synovial membrane from rheumatoid arthritis patients implanted subcutaneously into SCID mice. Overall these data provide more direct experimental evidence of the effects of BiP on the inflammatory process. It is also apparent that the anti-inflammatory characteristics of BiP are not due to any antigenic cross-reactivity. We suggest therefore, that the effects of BiP are not antigen specific and the immunomodulation induced may be relevant in all autoimmune diseases and that this strengthens the proposition that BiP may be useful for the treatment of these patients as well as those with rheumatoid arthritis (RA).

### Results

In the experiments described below BiP is only present during differentiation of PB monocytes to either, immature DC (iDC/iDC (BiP)) or mature DC (mDC)/mDC(BiP))

DC(BiP) showed phenotypic and functional differences from mDC.

Changes in iDC and mDC phenotype were monitored over a 7d differentiation period with maturation induced by LPS over the final 48 h. Intracellular indoleamine 2,3-dioxygenase (IDO) was consistently detected in more cells in the mDC(BiP) cultures (8.5±7 fold increase; range 2.0-20.0 fold) compared with control mDC (mDC, [mean ±standard deviation] 3.0±4.2% versus mDC(BiP), 14.1±7.1%, p=0.001, n=9) (FIG. 10A).

DC cultured in the presence of BiP sustained CD14 expression and failed to upregulate CD86 (FIG. 10BA-BF). This was a dose-dependent effect. In addition mDC(BiP) failed to mature, as determined by the upregulation of CD83, after addition of LPS (FIG. 10C). Other molecules expressed by mDC(BiP) that showed a reduced mean fluorescent intensity (MFI) included CD80 (p=0.07 ns)(Table A) and HLA-DR (p=0.04)(FIG. 10D) while the MFI for CD11c expressed by iDC was significantly increased (p=0.026) (Table A).

Table A: Surface expression of phenotypic markers: Purified monocytes were differentiated into immature dendritic cells (iDC) with GM-CSF and IL-4 for 7d in the presence or absence of BiP (20 μg/ml). LPS (500 ng/ml) was added for the final 2d to give mature DC(mDC). Cell surface expression of molecules was measured by immunofluorescence and flow cytometry. Results are expressed as mean and standard deviation (mean ±SD) of the mean fluorescent intensity (MFI) from 7 different experiments:

| | MFI | MFI | |
|---|---|---|---|
| | iDC | iDC(BiP) | |
| CD11c | 1511 ± 262 | 2246 ± 329 | p = 0.026 |
| | MFI | MFI | |
| | mDC | mDC(BiP) | |
| CD14 | 60 ± 19 | 366 ± 216 | p = 0.04 |
| CD86 | 343 ± 49 | 112 ± 14 | p = 0.04 |
| CD80 | 946 ± 166 | 395 ± 31 | p = 0.07 ns |
| CD1a | 1219 ± 400 | 685 ± 145 | p = 0.05 |
| HLA-DR | 2843 ± 168 | 1660 ± 167 | p = 0.044 |

In contrast a concomitant increase in cell surface expression of inhibitory molecule CD85j (ILT2) was observed (mDC, 72.5±6.4% versus mDC(BiP), 87.2±3.5%; p=0.008, n=4). (FIGS. 10E). Increased CD85j was also observed on iDC(BiP) particularly in conjunction with raised CD40 (iDC, 10±9% versus iDC(BIP), 26±19%; p=0.017, n=6)(Table 1). There appeared to be no difference in CD40 expression by mDC(BiP).

T cells co-cultured with BiP-treated DC show phenotypic and functional characteristics consistent with the generation of T regulatory cells.

Purified T cells showed phenotypic changes following co-culture with iDC(BiP) or mDC(BiP) when compared with co-culture with iDC or mDC.

a) Upregulation of T Regulatory Cell Markers

T cell surface expression for CD4CD25hiCD27hi (mDC, 0.8±0.2% versus mDC(BiP), 2.5±0.2%, p=0.016; n=5) (FIG. 11A) was significantly raised after contact with mDC(BiP).

b) Regulation of intracellular CTLA-4 by indolamine-2,3 dioxygenase containing dendritic cells.

CTLA-4 expression has been cited as a prerequisite for active Tregs (18). T cells that had been in contact with mDC(BiP) showed significant upregulation of intracellular CTLA-4 (FIG. 11B). To investigate whether increased CTLA-4 was a consequence of contact with IDO+ DC, the IDO inhibitor, 1 methyl tryptophan (1MT) was added to the cultures. Investigation of the expression of intracellular CTLA-4 following contact with mDC/mDC(BiP) in the presence of 1MT showed that inhibition of IDO function was associated with a significant reduction in T cell expression of CTLA-4 (p=0.013, n=3) (FIG. 11C). The functional relevance of this reduction in CTLA-4 was realised by the allogeneic responsiveness of those T cells in the presence of 1MT. The relative enhancement of response by T cells preincubated with mDC(BiP) and 1MT was significantly greater than for the corresponding mDC cultures with 1MT (p=0.033, n=3)(FIG. 11D)

c) T Cells Show Regulatory Activity Post Co-Culture with DC(BiP)

Washed T cells, taken from 4d co-cultures with either allogeneic or autologous mDC(BiP) inhibited the proliferation of autologous responder T cells to anti-CD3 antibody (FIG. 12A). The suppression of responder cell proliferation observed in the fully autologous cultures was greater than that induced by T cells co-cultured with allogeneic DC(BiP) (FIG. 12A, autologous, 63.8±13.7% inhibition versus allogeneic, 28.7±13.3% inhibition). Representative experiments of an autologous (FIG. 12B) or allogeneic (FIG. 12C) system show suppression of autologous responder T cells to anti-CD3 antibody in the presence of irradiated PBMC and T cells previously co-cultured with either iDC(BiP) or mDC(BiP) as indicated (ratio 1:1:1).

Allogeneic Response and Cytokine Production by Early BiP Treated DC Cultures

Allogeneic T cells stimulated by either iDC(BiP) or mDC(BiP) showed significantly reduced proliferation as shown above. Indeed, DC cultured with BiP for as little as 72 h already had this affect on T cell responses (FIG. 13A). The addition of a neutralizing anti-IL-10 antibody (FIG. 13A) or of the specific MAPK p38 inhibitor, SB203580, totally reversed BiP's inhibitory activity although the inhibition of MAPK ERK pathway using a specific inhibitor, PD98059, could not (FIG. 13B). That the inhibitory effect of BiP might be time dependent was illustrated by the failure of T cells to show a reduced allogeneic response to early DC when BiP had been added 24 h after GM-CSF and IL-4 (FIG. 13C).

Early DC(BiP) produced significantly more IL-10 than the control cultures (FIG. 13D). IL-10 production was not detected in the presence of neutralizing anti-IL-10 and SB203580. However PD98059 did not inhibit IL-10 production and therefore the T cell proliferative response remained depressed. The high production of IL-10 by DC(BiP) was sustained throughout the full 7d culture period (iDC, 71.2±54.2 versus iDC(BiP), 819.1±688 pg/ml, p=0.0136; mDC, 132.4±84.1 versus mDC(BiP), 970.1 ±684 pg/ml, p=0.0081, n=8)(FIG. 12E).

BiP causes increased intracellular IL-4 but reduced HLA-DR expression following incubation of human PBMC in the peritoneum of SCID mice.

The injection of human PBMC into the peritoneum of SCID mice has previously been used to investigate cell surface and intracellular cytokine changes during the development of human Th1 cells and more recently the role of IL-4 in Treg development (19). We have used the same model to investigate the effects of BiP. After 11 days very few human monocytes or B cells were detected in the population of cells retrieved from the peritoneal cavity. CD4+ cells from BiP treated samples expressed significantly increased IL-4 (BiP treated, 38.2±20% versus PBS, 20±11%; BiP n=5, PBS n=4; p=0.036) (FIG. 14A). Too few monocytes were retrieved to investigate IL-10 production by these cells. Confirmation of another phenotypic change already associated with BiP activation in vitro was noted, the downregulation of HLA-DR, albeit on CD4 T cells, and not antigen presenting cells (BiP treated, 50±6.2% versus PBS, 59±0.6%; BiP, n=5, PBS n=3; p=0.045) (FIG. 14B)

BiP Abrogates Inflammation in Human RASM Transplanted in SCID Mice

The RASM/SCID chimeric mouse is used to measure the efficacy of biologic therapies, such as anti-TNFα antibody, prior to human drug trials (20). Twelve days following intravenous injection of BiP into the RASM/SCID chimeric mice, the transplanted RASM was scored for characteristic features of inflammation according to the Rooney or Koizumi scoring systems. These include measurements of synovial hyperplasia, fibrosis, blood vessels, perivascular lymphocytes, lymphoid follicles, and diffuse infiltrating lymphocytes or synovial cells, palisading, giant cells, lymphocytes, granular tissue and fibrosis respectively. All measurements were significantly reduced in the transplanted RASM from the mice given intravenous BiP (Rooney: BiP, 16±6 versus HSA, 27±8.2; p=0.006; Koizumi: BiP; 6.1±2.6 versus HSA, 12±2.2; p<0.001).

Significantly reduced expression of CD86 and HLA-DR was seen in sections from the explants in the BiP-treated animals in conjunction with a decreased production of human TNFα and IL-6 (FIG. 15A) although no increase in IL-10 was detected. In the control mouse sera small, but easily detectable, quantities of human IL-6 were found while a significant reduction was observed in the circulation of mice given BiP (BiP, 0.5±0.2 pg/ml versus HSA, 1.9±1.9 pg/ml; p=0.028).

Despite the lack of evidence from the in vitro data of increased IL-10 at the chosen time-point we hypothesized that part, at least, of the therapeutic affect of BiP was mediated via IL-10. To test this hypothesis the effect of neutralizing anti-IL-10 antibody or isotype antibody control on the weight of the transplant was measured based on the observation that SM explants with reduced inflammation have reduced weight. The explants from the mice given HSA, the control protein, whether accompanied by anti-IL-10 antibody or the isotype control, did not differ in weight (HSA+anti-IL-10, 0.33±0.06 g; HSA+isotype, 0.27±0.07 g). In contrast, the explants from the mice given BiP+isotype control were significantly lighter, suggesting reduced cellularity, (BiP+isotype control, 0.07±0.04 g) than the weight of the BiP+anti-IL-10 explants. (BiP+anti-IL-10, 0.26±0.04 g; n=4 in each group; p=0.007 between the two BiP groups). There was no difference in weight between the 2 HSA groups and the group given BiP+anti-IL-10. Thus BiP reduces SM inflammation and this reduction is reversed by the administration of neutralizing anti-IL-10 antibody.

### Discussion of Example 10

We demonstrate the ability of extracellular BiP to down-regulate inflammatory immune responses through induction of IDO+ DC that generate CTLA-4+T cells with regulatory function. We have shown that the immunomodulatory and anti-DM inflammatory changes caused by BiP are similar in different in vitro and in vivo experimental models. This validates our invention regarding determination of BiP activity by assessment of various biomarker(s).

It is highly likely that the development of Treg is driven by BiP-induced changes to the DC phenotype acting to reduce antigen presentation efficacy in conjunction with increased IDO. High levels of IDO are known to enhance the tolerizing ability of DC (21) although the exact mechanism by which this is brought about is still being debated. At least two mechanisms of action are possible; either through the reduction in local levels of the essential amino acid tryptophan (21) or via inhibitory agents, such as kynurenines, down-stream products of tryptophan catabolism, that can induce apoptosis (21). Interpretation of the data can be difficult because the detection of intracellular IDO does not necessarily relate to functional activity. Enzymatic activity may therefore be investigated by monitoring the production of kynurenines or functionally, using the IDO inhibitor 1MT (22). In the context of this work, we have shown for the first time that mDC cultured with BiP consistently increased the number of IDO+competent mDC. Furthermore, we have shown that upregulation of intracellular CTLA-4 in allogeneic and autologous T cells following contact with mDC(BiP) may be inhibited by the addition of 1MT and that subsequent allogeneic proliferative responses are significantly improved. Munn and Mellor have already shown that CTLA-4.Ig can induce IDO in DC in vitro (21) and work done in macaques shows that CTLA-4 blockade downregulates IDO and TGFβ(23). We have now shown that BiP upregulates IDO in DC and direct contact between these DC and T cells, without additional BiP, induced T cell CTLA-4 expression completing this paracrine control loop.

Several candidates have been proposed as deactivation agents for human DC. Among these are IL-10(24), vitamin D3(25) and vasoactive intestinal peptide (VIP)(26). There is a general consensus that to generate anti-inflammatory DC there must be downregulation of the costimulatory molecules, CD86 and/or CD80, often in conjunction with upregulation of IL-10 and more recently, IDO, although IDO levels in the aforementioned cultures have not yet been reported. More subtle differences, however, may point to mechanistic diversity. For instance, addition of recombinant IL-10 reduced the MFI of HLA-DR expression (27), and suppressed CD86 expression (28). However, BiP caused profound decrease in CD86 expression and a reduced MFI of HLA-DR and CD80 expression while VIP caused a slight loss of CD86 and CD80 expression and had no affect on HLA-DR expression (29). So despite stimulation of the persistent production of IL-10 by VIP (29) or BiP, there are differences between the final phenotypes of mDC(BiP) and VIP-treated when compared with recombinant IL-10-treated mDC. Conversely, vitamin D3 treated DC, which produce little IL-10, induced high CD86 and low HLA-DR expression (25) but maintained features shared with mDC(BiP), namely, high CD14 and low CD83 expression. Collectively these findings suggest that DC cultured under these conditions fail to mature in the presence of LPS. In addition, iDC(BiP) and mDC(BiP) also show high expression of the inhibitory molecule, CD85j, associated with induction of tolerance (30). Co-ligation of HLA-DR enhances phosphorylation of the ITIM motif in the cytoplasmic tail of CD85j, thus BiP treatment should promote inhibition and/or tolerance. Expression on iDC(BiP) in conjunction with CD40 may further enhance the inhibitory potential of these cells, although this remains to be investigated.

To investigate whether IL-10 was a dominant suppressive mediator in our system, we decided to focus on its effect on antigen presentation in the early stages of DC differentiation. BiP treated DC in the early stages (3d) were unable to stimulate a mixed lymphocyte reaction of the same magnitude as control early DC. However, complete restoration of T cell proliferation to early DC(BiP) was observed following inhibition of IL-10 with SB 203580 or anti-IL-10. In contrast, inhibition of the MAPK ERK1/2 pathway, failed to restore the allogeneic reaction. Previous work, when recombinant IL-10 has been added to DC cultures has shown a temporal effect on antigen presenting efficiency (31) until ultimately no effect is seen after addition of IL-10 on day 6(27). We have been unable to alter DC development with BiP if it is added more than 24 h after the cultures are set up. It may be concluded therefore, that BiP has effects on DC development that are separate from and/or are additional to the IL-10 released from these cells under the influence of BiP.

In the present study there was a small but significant increase in the CD4+CD25hiCD27hi cells a recognised subset of regulatory T cells (32). CD27 has recently been identified as being a surface marker most closely associated with regulatory function for expanded Treg cells (32) but we are currently also investigating whether other regulatory T cell populations are induced. Since the exact phenotype of the inducible CD4 regulatory T cell is continually being refined we would just remark that the most notable evidence for an expansion of Treg is the increase of intracellular CTLA-4 in CD4+ cells following co-culture with DC(BiP). We found T cell proliferation to anti-CD3 antibody was inhibited by T cells pre-incubated with DC(BiP), in the complete absence of added BiP. This finding using human cells in vitro paralleled our observation, in mice, that lymph node and spleen cells given BiP parenterally will suppress CIA in recipient mice without the need for further BiP administration (16).

To investigate the development of BiP treated PBMC in vivo, a xenogeneic model using human PBMC injected into the peritoneal cavity of immune deficient SCID mice was used. Skapenko et al (2004)(19) and others have used this model to promote a Th1 cell immune response with characteristic upregulation of IFNγ and activation markers, CD25 and HLA-DR. Skapenko showed that endogenous production of IL-4 was crucial for the control of Th1 development. If endogenous IL-4 production was prevented then IFNγ production significantly increased. In contrast to their results, however, the addition of BiP to the PBMC skewed the development of T cells towards a Th2 phenotype with greatly increased numbers of IL-4+ T cells and a significantly reduced HLA-DR expression. Interestingly, recent work by Balic et al (2006) has shown that although in vitro work suggests that IL-4R signalling is essential for Th2 development their in vivo work has revealed a more fluid relationship. The initial development of IL-4 producing cells may be IL-4R independent but later, in competition with Th1 cell development, IL-10 is an absolute requirement for IL-4R dependent differentiation of Th2 cells (33). BiP stimulation of PBMC immediately prior to intraperitoneal injection would have ensured that these cells were producing much greater amounts of IL-10 than the control PBMC, although at 11d there were insufficient monocytes to measure IL-10. It might be hypothesized, though, that the IL-10 would drive the increase in IL-4+ T cells observed in the BiP treated PBMC although this remains to be formally tested. That IL-4 is a requirement for the expansion and development of Tregs in humans has previously been reported (19; 34) and IL-4 is also known to be a pre-requisite for the immunomodulatory function of BiP since no protective effect was shown by BiP when CIA was induced in the IL-4 knockout mouse (16).

Collectively the above data strongly support the hypothesis that BiP has anti-inflammatory properties not only by stimulating the secretion of IL-10 but also by the generation of Tregs. We decided, therefore, to put this hypothesis to the test by administering BiP to SCID mice following transplantation of RASM. Previously, this same SCID/RASM chimeric model has been used to predict the efficacy and therapeutic benefit of anti-TNFα monoclonal antibody (20) and MRA, an antibody that inhibits soluble circulating IL-6 receptor (35), prior to controlled clinical trials. In our tests, a single intravenous administration of BiP following vascularisation of the engrafted tissue suppressed inflammation in RASM as evidenced by the reduction in the histological inflammatory appearance of the explants using either the Rooney or the Koizumi scoring method. Further analysis showed that BiP had reduced the expression of the crucial molecules related to antigen presentation HLA-DR and CD86, directly corresponding to our human in vitro findings. Pro-inflammatory human cytokines TNFα, IL-1β and IL-6 were also reduced in the tissue and reflected the reduction of inflammation. In fact the beneficial affect of the inhibition of IL-6 production would be twofold, firstly, through reduction of inflammation and acute phase reactants and secondly, by inhibition of Th17 cell development for which IL-6 is a pre-requisite (36). Recent work, though, has shown that the requirement for IL-6 in the development of Th17 cells is more questionable in humans (37). However, despite the reduction in inflammatory cytokines little increase in IL-10 was detected although local production of IL-10 proved to be a necessary part of the anti-inflammatory mechanism of action of BiP since the administration of neutralizing anti-IL-10 antibody prevented the decrease in explant weight induced by BiP.

In conclusion, we have demonstrated that BiP modifies the maturation of DC in such a way that T cells, which come in contact with these DC, are driven towards a regulatory and/or TH2 cell phenotype. The above body of work supports our hypothesis that BiP is an immunomodulatory molecule that aids resolution of inflammation. It may thus be of potential therapeutic benefit in human inflammatory diseases such a RA.

### Materials and Methods for Example 10

Preparation of Recombinant Human BiP (rhuBiP)

BiP was prepared as described previously (38). The protein purity was greater than 95% as judged by silver staining and Associates of Cape Cod assessed endotoxin contamination at <0.06 ng/μg protein.

### Isolation of PBMC, T Cells and MO

PBMC were isolated from heparinised venous blood from healthy volunteers, after informed consent and approval of the project by the Guy's and St Thomas' Hospital Ethical Committee by density centrifugation over Lymphoprep (Nycomed-Pharma, Amersham, UK). T cells and MO were purified from PBMC by negative selection using the appropriate immunomagnetic kit (Dynal, Bromborough, UK).

### Differentiation of MO-Derived iDC and mDC:

Purified MO (>85% monocytes)(1.5.106/flask)(Corning Costar, High Wycombe, UK) were incubated in 5 ml of tissue culture medium (TCM)(RPMI 1640 medium (Sigma, Poole, UK) supplemented with 10% heat-inactivated foetal calf serum (FCS)(Life Technologies, Paisley, UK). iDC were generated by culturing MO with granulocyte macrophage-colony stimulating factor (GM-CSF) (1000 U/ml)(Novartis Research Institute, Vienna, Austria) and IL-4 (500 U/ml) (R&D Systems, Oxford, UK) for 3 or 7d, either alone or in the presence of rhuBiP (20 μg/ml). Cytokines and BiP were replenished every 2d. Mature DC were generated by addition of LPS (500 ng/ml) for the final 2d of a 7d culture.

### IL-10 Neutralisation and MAPK Inhibition.

To neutralize IL-10 within the cultures anti-IL-10 antibody (ATCC, Rockville, USA) was added at day 0 (5 μg/ml) to GM-CSF and IL-4 treated cultures in the presence and absence of BiP (20 μg/ml). Two specific cell-permeable inhibitors of the MAPK intracellular signalling pathways were used. SB203580 (SB)(10 μM)((Calbiochem, Darmstadt, Germany) inhibits p38 MAPK, and PD98059 (PD) (10 μM)(Calbiochem), an inhibitor of MAPK extracellular signal regulated kinase (ERK), were tested. MO were pre-treated with the inhibitors for 2 h before adding GM-CSF and IL-4 in the presence or absence of BiP.

### Determination of Regulatory T Cell Activity

Purified T cells (>95% T cells) (2.106/well) either autologous or allogeneic were cultured in the absence or presence of irradiated DC populations in 24-well flat-bottomed plates at a 1DC: 10 T cell ratio for 4d. T cells were isolated from the iDC/mDC allogeneic cultures and incubated with anti-HLA-DR antibody (clone L243, ATCC, Rockville USA, 1/20 dilution) and the DC removed with goat anti-mouse coated immunomagnetic beads (Dynal, Bromborough, UK). T cells (106/ml; 96 well plate) were then set up in an autologous culture with fresh responder T cells and irradiated PBMC (1:1:1 cell ratio) and cultured with or without anti-CD3 (clone OKT3, ATCC, Rockville, USA, 1/2000 dilution) for three days. The cells were pulsed with [3H] thymidine (Tdr)(0.2 μCi/well) (Pharmacia Biotech, Amersham, UK) for the final 18 h of culture.

### Quantification of Cytokines.

Supernatant samples were collected on day 1, 3 and 7 for iDC and from the final cultures of the T cells taken from the iDC/mDC-T cell allogeneic test system and set up with autologous cells to determine if there was any regulatory activity. The samples were aliquoted and frozen at −70° C. until required. Production of the pro-inflammatory cytokine, TNFα, and the anti-inflammatory cytokine, IL-10, was quantified by ELISA using paired antibodies and recombinant standards (PharMingen, Oxford, UK) according to the recommendations of the manufacturer. In addition some were analysed by cytometric bead assay (BD, Oxford, UK) according to the manufacturers instructions.

### Immunofluorescent Staining and Flow Cytometric Analysis

Immunofluorescent staining was performed, as described previously (39), on DC harvested at day 7, T cells after 4 days co-culture with DC. Saturating concentrations of fluorescein isothiocyanate (FITC)-, phycoerythrin (PE) or peridium chlorophyll (Per-CP)-conjugated mAb were added recognizing CD14, CD86, CD80, HLA-DR, CD11c, CD1a, CD83, CD85j, IL-10 receptor; purity of the negatively selected MO and T cells was checked using CD3, CD4, CD14, CD20 and CD57 (all from Becton Dickinson/PharMingen (BD), Oxford, UK). Intracellular staining was carried out following fixation of cells with 1% paraformaldehyde for 5 minutes. Cells were then washed with immunofluorescence buffer (PBS/0.1% BSA/0.05% azide) containing 0.3% saponin. 0.3% saponin was then present in all solutions thereafter. CD152/CTLA-4 (BD, Oxford UK) and IDO (Santa Cruz, Autogen Bioclear, UK) were intracellular stains. Isotype controls, IgG1 and IgG2a (BD, Oxford, UK) were used in parallel. Cells were analysed on a FACScan/FACScalibur cytometers using Cellquest software (BD, Oxford, UK).

### SCID Mouse Studies

All experiments had the relevant ethical committee approval.

### Intraperitoneal Injection of Human PBMC:

SCID mice were given an intraperitoneal injection of human PBMC (50.106/mouse) in the presence or absence of BiP (100 μg/mouse) and sacrificed 11d later when the peritoneum was washed out with PBS and the remaining cells collected for analysis. Cells were examined for the intracellular presence of IL-4 or IFNγ and for cell surface molecules such as HLA-DR and CD86.

### Preparation of SCID-RASM Mouse:

This study used the previously developed SCID-HuRAg murine model (20). Briefly, pannus tissue, consisting of the leading edge of the invading synovial membrane into articular cartilage and bone, was collected as a single mass from RA patients at the time of arthroplastic surgery with full ethical approval. A block of 4-8 mm in diameter was implanted subcutaneously into the back of 6- to 7-week-old male SCID mice (CB.17/Icr; Charles River Japan, Tokyo, Japan), which had been bred under specific pathogen-free conditions. Successful implantation of human RA tissue was assessed visually 4 weeks after implantation, after which therapeutic manipulation of the mice was undertaken. BiP (10 μg/mouse) or HSA (10 μg/mouse) was administrated by single intravenous injection. The mice were sacrificed 12 days after injection to allow removal of the implanted tissue.

### Scoring of the Degree of Synovial Inflammation and Inflammatory Cell Infiltrate

The degree of synovial inflammation of the implanted tissue was assessed as previously described. The histological findings were scored according to two different scoring systems: Koizumi's (40) or Rooney's method (41).

### Immunohistological Examination

Paraffin embedded tissue sections were used for immunostaining for CD86 and HLA-DR. Frozen tissue sections were stained for the detection of cytokines (TNF, IL-1, IL-6, and IL-10). The tissue sections were blocked for endogenous peroxidase activity with 0.3% hydrogen peroxide in methanol for 15 min. Non-specific antibody binding was blocked with 10% normal goat serum for 1 hr at room temperature. The sections were incubated with specific antibody or normal IgG for 1 hr at 37° C. The sections incubated with anti-CD86 antibody were treated with biotinylated secondary antibody, then visualised using a kit (Vectastatin ABC kit, Vector) with 3-amino-n-ethylcarbazole (Nichirei, Tokyo) as a substrate. The other sections were treated with peroxidase-conjugated anti-mouse IgG (Histofine Simple Stain MAX PO; Nichirei, Tokyo) for 30 min at room temperature and developed with 3-amino-n-ethylcarbazole (Nichirei, Tokyo) for visualization. Sections were counterstained with Mayer haematoxylin for 10 sec and mounted in aqueous permanent mounting solution (Nichirei, Tokyo).

### Measurement of Human IL-4, IL-6 and IL-10 in Mouse Serum

The serum levels of human IL-4, IL-6 and IL-10 were measured by a quantitative sandwich enzyme immunoassay technique (Quantikine HS, R&D systems) according to the manufacturer's instructions.

### Statistical Analysis

All experiments were performed at least three times. Data were compared using the student's T test and expressed as mean ±SD in the text.

### REFERENCES TO EXAMPLE 10

1. Maldonado-Lopez, R. and Moser, M. 2001. Dendritic cell subsets and the regulation of Th1/Th2 responses. Semin. Immunol. 13:275-282.
2. Weaver, C. T., Hatton, R. D., Mangan, P. R., and Harrington, L. E. 2007. IL-17 family cytokines and the expanding diversity of effector T cell lineages. Annu. Rev. Immunol. 25:821-852.
3. Romagnani, S. 2006. Regulation of the T cell response. Clin. Exp. Allergy 36:1357-1366.
4. Gonzalez-Rey, E., Chorny, A., Fernandez-Martin, A., Ganea, D., and Delgado, M. 2006. Vasoactive intestinal peptide generates human tolerogenic dendritic cells that induce CD4 and CD8 regulatory T cells. Blood 107:3632-3638.
5. Gething, M. J. 1999. Role and regulation of the ER chaperone BiP. Semin. Cell Dev. Biol. 10:465-472.
6. Asea, A., Kraeft, S. K., Kurt-Jones, E. A., Stevenson, M. A., Chen, L. B., Finberg, R. W., Koo, G. C., and Calderwood, S. K. 2000. HSP70 stimulates cytokine production through a CD14-dependant pathway, demonstrating its dual role as a chaperone and cytokine. Nat. Med. 6:435-442.
7. Asea, A., Rehli, M., Kabingu, E., Boch, J. A., Bare, O., Auron, P. E., Stevenson, M. A., and Calderwood, S. K. 2002. Novel signal transduction pathway utilized by extracellular HSP70: role of toll-like receptor (TLR) 2 and TLR4. J. Biol. Chem. 277:15028-15034.
8. Wang, Y., Kelly, C. G., Karttunen, J. T., Whittall, T., Lehner, P. J., Duncan, L., MacAry, P., Younson, J. S., Singh, M., Oehlmann, W. et al. 2001. CD40 is a cellular receptor mediating mycobacterial heat shock protein 70 stimulation of CC-chemokines. Immunity. 15:971-983.
9. Delneste, Y., Magistrelli, G., Gauchat, J., Haeuw, J., Aubry, J., Nakamura, K., Kawakami-Honda, N., Goetsch, L., Sawamura, T., Bonnefoy, J. et al. 2002. Involvement of LOX-1 in dendritic cell-mediated antigen cross-presentation. Immunity. 17:353-362.
10. Binder, R. J., Han, D. K., and Srivastava, P. K. 2000. CD91: a receptor for heat shock protein gp96. Nat. Immunol 1:151-155.
11. Binder, R. J., Harris, M. L., Menoret, A., and Srivastava, P. K. 2000. Saturation, competition, and specificity in interaction of heat shock proteins (hsp) gp96, hsp90, and hsp70 with CD11b+ cells. J. Immunol. 165:2582-2587.
12. Matzinger, P. 2002. The danger model: a renewed sense of self. Science 296:301-305.
13. Habich, C., Baumgart, K., Kolb, H., and Burkart, V. 2002. The receptor for heat shock protein 60 on macrophages is saturable, specific, and distinct from receptors for other heat shock proteins. J. Immunol. 168:569-576.
14. Corrigall, V. M., Bodman-Smith, M. D., Brunst M, Cornell H, and Panayi, G. S. 2004. The stress protein, BiP, stimulates human peripheral blood mononuclear cells to express an anti-inflammatory cytokine profile and to inhibit antigen presenting cell function: relevance to the treatment of inflammatory arthritis. Arthritis Rheum. 50:1167-1171.
15. Bodman-Smith, M. D., Corrigall, V. M., Kemeny, D. M., and Panayi, G. S. 2003. BiP, a putative autoantigen in rheumatoid arthritis, stimulates IL-10-producing CD8-positive T cells from normal individuals. Rheumatology. (Oxford) 42:637-644.
16. Brownlie, R. J., Myers, L. K., Wooley, P. H., Corrigall, V. M., Bodman-Smith, M. D., Panayi, G. S., and Thompson, S. J. 2006. Treatment of murine collagen-induced arthritis by the stress protein BiP via interleukin-4-producing regulatory T cells: a novel function for an ancient protein. Arthritis Rheum. 54:854-863.
17. Panayi, G. S., Corrigall, V. M., and Henderson, B. 2004. Stress cytokines: pivotal proteins in immune regulatory networks; Opinion. Curr. Opin. Immunol. 16:531-534.
18. Dieckmann, D., Plottner, H., Berchtold, S., Berger, T., and Schuler, G. 2001. Ex vivo isolation and characterization of CD4(+)CD25(+) T cells with regulatory properties from human blood. J. Exp. Med. 193:1303-1310.
19. Skapenko, A., Niedobitek, G. U., Kalden, J. R., Lipsky, P. E., and Schulze-Koops, H. 2004. Generation and regulation of human Th1-biased immune responses in vivo: a critical role for IL-4 and IL-10. J. Immunol. 172:6427-6434.
20. Matsuno, H., Yudoh, K., Katayama, R., Nakazawa, F., Uzuki, M., Sawai, T., Yonezawa, T., Saeki, Y., Panayi, G. S., Pitzalis, C. et al. 2002. The role of TNF-alpha in the pathogenesis of inflammation and joint destruction in rheumatoid arthritis (RA): a study using a human RA/SCID mouse chimera. Rheumatology (Oxford) 41:329-337.
21. Mellor, A. L. and Munn, D. H. 2004. IDO expression by dendritic cells: tolerance and tryptophan catabolism. Nat. Rev. Immunol. 4:762-774.
22. Hwang, S. L., Chung, N. P., Chan, J. K., and Lin, C. L. 2005. Indoleamine 2,3-dioxygenase (IDO) is essential for dendritic cell activation and chemotactic responsiveness to chemokines. Cell Res. 15:167-175.
23. Hryniewicz, A., Boasso, A., Edghill-Smith, Y., Vaccari, M., Fuchs, D., Venzon, D., Nacsa, J., Betts, M. R., Tsai, W. P., Heraud, J. M. et al. 2006. CTLA-4 blockade decreases TGF-beta, IDO, and viral RNA expression in tissues of SIVmac251-infected macaques. Blood 108:3834-3842.
24. Rutella, S., Danese, S., and Leone, G. 2006. Tolerogenic dendritic cells: cytokine modulation comes of age. Blood 108:1435-1440.
25. Canning, M. O., Grotenhuis, K., de Wit, H., Ruwhof, C., and Drexhage, H. A. 2001. 1-alpha, 25-Dihydroxyvitamin D3 (1,25(OH)(2)D(3)) hampers the maturation of fully active immature dendritic cells from monocytes. Eur. J. Endocrinol. 145:351-357.
26. Delgado, M., Gonzalez-Rey, E., and Ganea, D. 2006. Vasoactive intestinal peptide: the dendritic cell--> regulatory T cell axis. Ann. N.Y. Acad. Sci. 1070:233-238.
27. Velten, F. W., Duperrier, K., Bohlender, J., Metharom, P., and Goerdt, S. 2004. A gene signature of inhibitory MHC receptors identifies a BDCA3(+) subset of IL-10-induced dendritic cells with reduced allostimulatory capacity in vitro. Eur. J. Immunol. 34:2800-2811.
28. Figueroa-Vega, N., Galindo-Rodriguez, G., Bajana, S., Portales-Perez, D., Abud-Mendoza, C., Sanchez-Torres, C., and Gonzalez-Amaro, R. 2006. Phenotypic analysis of IL-10-treated, monocyte-derived dendritic cells in patients with systemic lupus erythematosus. Scand. J. Immunol. 64:668-676.
29. Delgado, M., Gonzalez-Rey, E., and Ganea, D. 2005. The neuropeptide vasoactive intestinal peptide generates tolerogenic dendritic cells. J. Immunol. 175:7311-7324.
30. Schuler, G., Schuler-Thumer, B., and Steinman, R. M. 2003. The use of dendritic cells in cancer immunotherapy. Curr. Opin. Immunol. 15:138-147.
31. Allavena, P., Piemonti, L., Longoni, D., Bernasconi, S., Stoppacciaro, A., Ruco, L., and Mantovani, A. 1998. IL-10 prevents the differentiation of monocytes to dendritic cells but promotes their maturation to macrophages. Eur. J. Immunol. 28:359-369.
32. Duggleby, R. C., Shaw, T. N., Jarvis, L. B., Kaur, G., and Gaston, J. S. 2007. CD27 expression discriminates between regulatory and non-regulatory cells after expansion of human peripheral blood CD4+ CD25+ cells. Immunology 121:129-139.
33. Balic, A., Harcus, Y. M., Taylor, M. D., Brombacher, F., and Maizels, R. M. 2006. IL-4R signaling is required to induce IL-10 for the establishment of T(h)2 dominance. Int. Immunol. 18:1421-1431.
34. Mendel, I. and Shevach, E. M. 2002. The IL-10-producing competence of Th2 cells generated in vitro is IL-4 dependent. Eur. J. Immunol. 32:3216-3224.
35. Ito, H.2005. Treatment of Crohn's disease with anti-IL-6 receptor antibody. J. Gastroenterol. 40 Suppl 16:32-34.
36. Afzali, B., Lombardi, G., Lechler, R. I., and Lord, G. M. 2007. The role of T helper 17 (Th17) and regulatory T cells (Treg) in human organ transplantation and autoimmune disease. Clin. Exp. Immunol. 148:32-46.
37. Chen, Z., Tato, C. M., Muul, L., Laurence, A., and O'shea, J. J. 2007. Distinct regulation of interleukin-17 in human T helper lymphocytes. Arthritis Rheum. 56:2936-2946.
38. Corrigall, V. M., Bodman-Smith, M. D., Fife, M. S., Canas, B., Myers, L. K., Wooley, P., Soh, C., Staines, N. A., Pappin, D. J., Berlo, S. E. et al. 2001. The human endoplasmic reticulum molecular chaperone BiP is an autoantigen for rheumatoid arthritis and prevents the induction of experimental arthritis. J. Immunol. 166:1492-1498.
39. Iannone, F., Corrigall, V. M., Kingsley, G. H., and Panayi, G. S. 1994. Evidence for the continuous recruitment and activation of T cells into the joints of patients with rheumatoid arthritis. Eur. J. Immunol. 24:2706-2713.
40. Koizumi, F., Matsuno, H., Wakaki, K., Ishii, Y., Kurashige, Y., and Nakamura, H. 1999. Synovitis in rheumatoid arthritis: scoring of characteristic histopathological features. Pathol. Int. 49:298-304.
41. Rooney, M., Condell, D., Quinlan, W., Daly, L., Whelan, A., Feighery, C., and Bresnihan, B. 1988. Analysis of the histologic variation of synovitis in rheumatoid arthritis. Arthritis Rheum. 31:956-963.

### REFERENCES

(1) Corrigall V M, Bodman-Smith M D, Fife M S, Canas B, Myers L K, Wooley P et al. The human endoplasmic reticulum molecular chaperone BiP is an autoantigen for rheumatoid arthritis and prevents the induction of experimental arthritis. J Immunol 2001; 166(3):1492-1498.
(2) Bodman-Smith M D, Corrigall V M, Kemeny D M, Panayi G S. BiP, a putative autoantigen in rheumatoid arthritis, stimulates IL-10-producing CD8-positive T cells from normal individuals. Rheumatology (Oxford) 2003; 42(5):637-644.
(3) Corrigall V M, Bodman-Smith M D, Brunst M, Cornell H, Panayi G S. The stress protein, BiP, stimulates human peripheral blood mononuclear cells to express an anti-inflammatory cytokine profile and to inhibit antigen presenting cell function: relevance to the treatment of inflammatory arthritis. Arthritis Rheum 2004; 50:1167-1171.
(4) Miossec P, Chomarat P, Dechanet J, Moreau J F, Roux J P, Delmas P et al. Interleukin-4 inhibits bone resorption through an effect on osteoclasts and proinflammatory cytokines in an ex vivo model of bone resorption in rheumatoid arthritis. Arthritis Rheum 1994; 37(12):1715-1722.
(5) Yang S Y, Wu B, Mayton L, Mukheijee P, Robbins P D, Evans C H et al. Protective effects of IL-1Ra or vIL-10 gene transfer on a murine model of wear debris-induced osteolysis. Gene Ther 2004; 11(5):483-491.
(6) Vittecoq O, Corrigall V M, Bodman-Smith M D, Panayi G S. The molecular chaperone BiP (GRP78) inhibits the differentiation of normal human monocytes into immature dendritic cells. Rheumatology (Oxford) 2003; 42 suppl:43.

All publications mentioned in the above specification are herein incorporated by reference. Various modifications and variations of the described methods and system of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in biochemistry and molecular biology or related fields are intended to be within the scope of the following claims.

We claim:

1. A method for determining the functionality of BiP in vivo, comprising the steps of:
(i) providing a sample from a subject;
(ii) contacting the sample with BiP; and
(iii) determining the presence of a marker in the sample, wherein said marker is selected from the group consisting of:
(a) an increase in the number of cells expressing indoleamine 2,3-dioxygenase (IDO);
(b) an increase in the number of cells expressing CD8CD122;
(c) an increase in the number of cells expressing CD4CD25hiCD27hi;
(d) an increase in the number of cells expressing CD4CD25hi FoxP3;
(e) an increase in the number of cells expressing intracellular CTLA-4;
(f) an increase in the number of cells expressing CCR4;
(g) a decrease in the number of cells expressing CD86;
(h) an increase in the number of cells expressing cell surface CD85j;
(i) an decrease in the number of cells expressing CD83;
(j) an increase or no change in the number of cells expressing CD14;
(k) a decrease in the number of T regulatory cell or dendritic cells expressing HLA-DR or Class II Major Histocompatibility Complex antigen;
(l) a decrease in the number of cells expressing CD80;
(m) an increase in the number of cells expressing CD40;
(n) a lower CD11a mean fluorescent intensity;
(O) a higher CD11c mean fluorescent intensity;
(p) dendritic cells that do not present antigen and do not stimulate an allogeneic lymphocyte reaction;
(q) an increase in the number of T regulatory cell or dendritic cells expressing IL-10;
(r) an increase in the number of human T cells cultured in the peritoneum of SCID mice that show IL-4, IL-5 and/or IL-10 expression and/or a decrease in the number of cells expressing interferon-γ;
(s) no upregulation of IFNγ producing cells when stimulated with a T cell mitogen;
(t) a decrease in the concentration of circulating IL-6; and
(u) a decrease or downregulation of IL-8;
or a combination comprising at least 2 of said markers;
wherein the difference is measured in comparison to a sample or a cell that has not been induced with BiP, and
wherein the presence of the marker(s) in the sample is indicative that BiP is functional in vivo.

2. A method according to claim 1 wherein said marker is selected from the group consisting of:
(a) an increase in the number of cells expressing indoleamine 2,3-dioxygenase (IDO);
(b) an increase in the number of cells expressing CD8CD122;
(c) an increase in the number of cells expressing CD4CD25hiCD27hi;
(d) an increase in the number of cells expressing CD4CD25hi FoxP3;
(e) an increase in the number of cells expressing intracellular CTLA-4;
(f) an increase in the number of cells expressing CCR4;
(g) a decrease in the number of cells expressing CD80;
(h) lower CD11a mean fluorescent intensity;
(i) higher CD11c mean fluorescent intensity;
(j) dendritic cells that do not present antigen and do not stimulate an allogeneic lymphocyte reaction;
(k) an increase in the number of human T cells cultured in the peritoneum of SCID mice that show IL-4, IL-5 and/or IL-10 expression and/or a decrease in the number of cells expressing interferon-γ;
(l) no upregulation of IFNγ producing cells when stimulated with a T cell mitogen; and
(m) a decrease in the concentration of circulating IL-6;
or a combination comprising at least 2 of said markers;
wherein the difference is measured in comparison to a sample or a cell that has not been induced with BiP; and
wherein the presence of the marker(s) in the sample is indicative that BiP is functional in vivo.

3. A method according to claim 1 wherein the marker is CTLA-4 and/or IDO.

4. The method according to claim 1 wherein the marker is IL-8.

5. The method according to claim 1, wherein the subject has been administered with BiP.

6. The method according to claim 1, wherein the sample or the cell that has not been induced with BiP is obtained or obtainable from the subject before the subject has been administered with BiP.

7. The method according to claim 1, wherein the presence of one or more markers in the sample is determined by transferring the sample intraperitoneally into severe combined immunodeficient (SCID) mice.

8. The method according claim 1, wherein the cell expressing the marker set forth in (b), (c), (d) (e), (f), (g), (h), (i), (j), (k), (l) and/or (m) is a regulatory T-cell or a dendritic cell.

9. The method according to claim 2, wherein the cell expressing the marker set forth in (b), (c), (d) (e), (f), (g) and/or (h) is a regulatory T-cell or a dendritic cell.

10. The method according to claim 1, wherein marker (r) is measured about 11 days after contacting the sample with BiP.

11. The method according to claim 2 wherein marker (l) is measured about 11 days after contacting the sample with BiP.

12. A kit for determining the functionality of BiP in vivo comprising a plurality of labelled antibodies, wherein said labelled antibodies are selected from labelled antibodies which specifically hybridise to one or more of CD14, CD80, CD83, CD86, CD85j, HLA-DR, CD11, CD1, CD4, CD8, CD25, CD27, CD122, CTLA-4, FoxP3, IL-10, IL-4, IL-5, IL-6, IFNγ, IDO, CCR-4, CD40, and IL-8.

13. A kit according to claim 12, wherein said labelled antibodies are selected from labelled antibodies which specifically hybridise to one or more of CD80, CD11, CD1, CD4, CD8, CD25, CD27, CD122, CTLA-4, FoxP3, IL-10, IL-4, IL-5, IL-6, IFNγ, IDO and CCR-4.

14. The kit according to claim 12, wherein the antibody is a monoclonal antibody.

15. The kit according to claim 12, wherein the label is a florescent label.

16. The kit according to claim 12, wherein said kit comprises concanavalin A.

17. The kit according to claim 12, wherein the kit comprises anti-CD3 and/or CD28 beads.

18. The kit according to claim 12, wherein the kit comprises immunofluorescent buffer and/or saponin solution.

19. Use of a marker for determining the functionality of BiP in vivo, wherein said marker is selected from the group consisting of:
(a) an increase in the number of cells expressing indoleamine 2,3-dioxygenase (IDO);
(b) an increase in the number of cells expressing CD8CD122;
(c) an increase in the number of cells expressing CD4CD25hiCD27hi;
(d) an increase in the number of cells expressing CD4CD25hi FoxP3;
(e) an increase in the number of cells expressing intracellular CTLA-4;
(f) an increase in the number of cells expressing CCR4;
(g) a decrease in the number of cells expressing CD86;
(h) an increase in the number of cells expressing cell surface CD85j;
(i) an decrease in the number of cells expressing CD83;
(j) an increase or no change in the number of cells expressing CD14;
(k) a decrease in the number of T regulatory cell or dendritic cells expressing HLA-DR or Class II Major Histocompatibility Complex antigen;
(l) a decrease in the number of cells expressing CD80;
(m) an increase in the number of cells expressing CD40;
(n) a lower CD11a mean fluorescent intensity;
(O) a higher CD11c mean fluorescent intensity;
(p) dendritic cells that do not present antigen and do not stimulate an allogeneic lymphocyte reaction;
(q) an increase in the number of T regulatory cell or dendritic cells expressing IL-10;
(r) an increase in the number of human T cells cultured in the peritoneum of SCID mice that show IL-4, IL-5 and/or IL-10 expression and/or a decrease in the number of cells expressing interferon-γ;
(s) no upregulation of IFNγ producing cells when stimulated with a T cell mitogen;
(t) a decrease in the concentration of circulating IL-6; and
(u) a decrease or downregulation of IL-8;
or a combination comprising at least 2 of said markers;
wherein the difference is measured in comparison to a cell that has not been induced by BiP; and
wherein the presence of the marker(s) in the sample is indicative that BiP is functional in vivo.

20. Use according to claim 19, wherein said marker is selected from the group consisting of:
(a) an increase in the number of cells expressing indoleamine 2,3-dioxygenase (IDO);
(b) an increase in the number of cells expressing CD8CD122;
(c) an increase in the number of cells expressing CD4CD25hiCD27hi;
(d) an increase in the number of cells expressing CD4CD25hi FoxP3;
(e) an increase in the number of cells expressing intracellular CTLA-4;
(f) an increase in the number of cells expressing CCR4;
(g) a decrease in the number of cells expressing CD80;
(h) lower CD11a mean fluorescent intensity;
(i) higher CD11c mean fluorescent intensity;
(j) dendritic cells that do not present antigen and do not stimulate an allogeneic lymphocyte reaction;
(k) an increase in the number of human T cells cultured in the peritoneum of SCID mice that show IL-4, IL-5 and/or IL-10 expression and/or a decrease in the number of cells expressing interferon-γ;
(l) no upregulation of IFNγ producing cells when stimulated with a T cell mitogen; and
(m) a decrease in the concentration of circulating IL-6;
or a combination comprising at least 2 of said markers.
